SEQUENCE LISTING (170 sequences)

SEQ ID NO: 1 - DNA, 41 bp, Artificial Sequence
  Synthetic construct
acacacacacaacgaacggcagttactccggcccccctgca

SEQ ID NO: 2 - DNA, 40 bp, Artificial Sequence
  Synthetic construct
acacccccaccacccttgcacacatcacactccacacgca

SEQ ID NO: 3 - DNA, 40 bp, Artificial Sequence
  Synthetic construct
accaccacaccacacccgaccctcctcctaccatcccacc

SEQ ID NO: 4 - DNA, 40 bp, Artificial Sequence
  Synthetic construct
acacgccacacacaacgaaactaacgccgggatacgccac

SEQ ID NO: 5 - DNA, 40 bp, Artificial Sequence
  Synthetic construct
gcacacaccaaacattagacacacaagaacagggcggccc

SEQ ID NO: 6 - DNA, 40 bp, Artificial Sequence
  Synthetic construct
acgccaccacacacacagcctaccaccacgcggaagctcc

SEQ ID NO: 7 - DNA, 40 bp, Artificial Sequence
  Synthetic construct
gcacaaccaacaacagacaacccacccacccacgccgttc

SEQ ID NO: 8 - DNA, 40 bp, Artificial Sequence
  Synthetic construct
gcacaccacacaaccaaaagcaaggaaccaatacccaccg

SEQ ID NO: 9 - DNA, 40 bp, Artificial Sequence
  Synthetic construct
gcacacacacacattacacagtgatagaccacccaaatcc

SEQ ID NO: 10 - DNA, 40 bp, Artificial Sequence
  Synthetic construct
gcgccacacacacatacacacactagcagaaacacttcgc

SEQ ID NO: 11 - DNA, 40 bp, Artificial Sequence
  Synthetic construct
cacacacacacacacttcttcgggggaaccagtacgcggt

SEQ ID NO: 12 - DNA, 40 bp, Artificial Sequence
  Synthetic construct
ggggggaagggtgggactttaaccatacttcggtggcctg

SEQ ID NO: 13 - DNA, 40 bp, Artificial Sequence
  Synthetic construct
ggggggcagggtgggactttaaccatacttcggtggcatg

SEQ ID NO: 14 - DNA, 40 bp, Artificial Sequence
  Synthetic construct
ggggggaagggtgggactttaaccatacttcggtggaatg

SEQ ID NO: 15 - DNA, 40 bp, Artificial Sequence
  Synthetic construct
ggggggaagggcgggactttaaccatacttcggtggcatg

SEQ ID NO: 16 - DNA, 40 bp, Artificial Sequence
  Synthetic construct
tttgggagggttgaggtgggggaggtggaggtagttagag

SEQ ID NO: 17 - DNA, 40 bp, Artificial Sequence
  Synthetic construct
acacacaccacgcacacccacactctggccgggacgacag

SEQ ID NO: 18 - DNA, 40 bp, Artificial Sequence
  Synthetic construct
accacaaccccacgtcacactggctacacaccaacgctcc

SEQ ID NO: 19 - DNA, 40 bp, Artificial Sequence
  Synthetic construct
acaccacagccgcaaacacacacgcacaactccgatcgtc

SEQ ID NO: 20 - DNA, 40 bp, Artificial Sequence
  Synthetic construct
caccgcaggatactacacaccagcacacagccactacaca

SEQ ID NO: 21 - DNA, 40 bp, Artificial Sequence; identical to SEQ ID NO: 9
  Synthetic construct

SEQ ID NO: 22 - DNA, 40 bp, Artificial Sequence
  Synthetic construct
accaccaaccacgaggcacctccaccacagcgcttccacc

SEQ ID NO: 23 - DNA, 40 bp, Artificial Sequence; identical to SEQ ID NO: 5
  Synthetic construct

SEQ ID NO: 24 - DNA, 40 bp, Artificial Sequence
  Synthetic construct
caacaagcacacactcactacacactcacacacgccttgg

SEQ ID NO: 25 - DNA, 40 bp, Artificial Sequence
  Synthetic construct
gaccacggtacagacagacacacacacagacgtttcccac

SEQ ID NO: 26 - DNA, 40 bp, Artificial Sequence
  Synthetic construct
ggtggagggtgggaagagggatgaggtgtgtaaggggggg

SEQ ID NO: 27 - DNA, 40 bp, Artificial Sequence
  Synthetic construct
ggtggaggttatatactatccgggcgggtttagggggggt

SEQ ID NO: 28 - DNA, 40 bp, Artificial Sequence
  Synthetic construct
ggggttgggggaggttatgactttttgggggggggacttc

SEQ ID NO: 29 - DNA, 40 bp, Artificial Sequence
  Synthetic construct
ggtggaggttatatactatccgggtgggtttagggggggt

SEQ ID NO: 30 - DNA, 40 bp, Artificial Sequence
  Synthetic construct
ggggtgggttaactttttgggggggttgttattttgggga

SEQ ID NO: 31 - DNA, 40 bp, Artificial Sequence
  Synthetic construct
gctagggcgggtggagggtagtttgcgcgggggtggaggt

SEQ ID NO: 32 - DNA, 40 bp, Artificial Sequence
  Synthetic construct
gggggggtagtaactgtgggtgggggaggtggaggttata

SEQ ID NO: 33 - DNA, 40 bp, Artificial Sequence
  Synthetic construct
gggggagggggagaggagaggaaggtggtaaagagtgggg

SEQ ID NO: 34 - DNA, 40 bp, Artificial Sequence
  Synthetic construct
ggggtgggttaaatttttgggggggttgttattttgggga

SEQ ID NO: 35 - DNA, 40 bp, Artificial Sequence
  Synthetic construct
tttgggagggttgaggtgggggaggaggaggtagttagag

SEQ ID NO: 36 - DNA, 40 bp, Artificial Sequence
  Synthetic construct
tttgggcgggttgaggtgggggaggaggaggtagttagag

SEQ ID NO: 37 - DNA, 40 bp, Artificial Sequence
  Synthetic construct
tttgggagggttgaggcgggggaggaggaggtagttagag

SEQ ID NO: 38 - DNA, 40 bp, Artificial Sequence
  Synthetic construct
catggcggggggggggggagaaggggggggggggggtttt

SEQ ID NO: 39 - DNA, 40 bp, Artificial Sequence
  Synthetic construct
tacgggtggaggggggggcggttggttgtagttatttggt

SEQ ID NO: 40 - DNA, 40 bp, Artificial Sequence
  Synthetic construct
tttgggggggttgaggtgggggaggaggaggtagttagag

SEQ ID NO: 41 - DNA, 40 bp, Artificial Sequence
  Synthetic construct
gctagggcgggtggagggtagtttgcgcgggggcggaggt

SEQ ID NO: 42 - DNA, 40 bp, Artificial Sequence
  Synthetic construct
tttgggagggttgaggtgggggaggcggaggtagttagag

SEQ ID NO: 43 - DNA, 40 bp, Artificial Sequence
  Synthetic construct
tacgggtggaggggggggcggttggttgcagttatttggt

SEQ ID NO: 44 - DNA, 40 bp, Artificial Sequence
  Synthetic construct
tttgggagggttgaggtgggggagggggaggtagttagag

SEQ ID NO: 45 - DNA, 40 bp, Artificial Sequence
  Synthetic construct
tttggcagggttgaggtgggggaggaggaggtagttagag

SEQ ID NO: 46 - DNA, 40 bp, Artificial Sequence
  Synthetic construct
gcggggggtggaaagaggatgaaggggggtggattttggg

SEQ ID NO: 47 - DNA, 40 bp, Artificial Sequence
  Synthetic construct
catggcgggggggggggcagaaggggggggggggggtttt

SEQ ID NO: 48 - DNA, 40 bp, Artificial Sequence
  Synthetic construct
catggcgggggggggggcagaaggggggggggggggtttg

SEQ ID NO: 49 - DNA, 40 bp, Artificial Sequence
  Synthetic construct
catggcggggggggggggagaaggggggggggggggtttg

SEQ ID NO: 50 - DNA, 40 bp, Artificial Sequence
  Synthetic construct
ggtggcgggtgggaagagggatgaggtgtgtaaggggggg

SEQ ID NO: 51 - DNA, 40 bp, Artificial Sequence
  Synthetic construct
ggtggcggttctatactatccgggtgggtttagggggggt

SEQ ID NO: 52 - DNA, 40 bp, Artificial Sequence
  Synthetic construct
ggtggaggttctatactatccgggtgggtttagggggggt

SEQ ID NO: 53 - DNA, 40 bp, Artificial Sequence
  Synthetic construct
gggggggtcgtaactgtgggtgggggaggtggaggttata

SEQ ID NO: 54 - DNA, 40 bp, Artificial Sequence
  Synthetic construct
ggggtgggttcactttttgggggggttgttcttttgggga

SEQ ID NO: 55 - DNA, 40 bp, Artificial Sequence
  Synthetic construct
tgggagggttggagggggtggaggtgagggtttattcaga

SEQ ID NO: 56 - DNA, 40 bp, Artificial Sequence
  Synthetic construct
tttgggagggctgaggtgggggaggaggaggtagttagag

SEQ ID NO: 57 - DNA, 40 bp, Artificial Sequence
  Synthetic construct
tttgggagggtggaggtgggggaggcggaggtagttagag

SEQ ID NO: 58 - DNA, 40 bp, Artificial Sequence
  Synthetic construct
tttgggagggttgaggagggggaggaggaggtagttagag

SEQ ID NO: 59 - DNA, 40 bp, Artificial Sequence
  Synthetic construct
tttgggagggtcgaggtgggggagggggaggtagttagag

SEQ ID NO: 60 - DNA, 40 bp, Artificial Sequence
  Synthetic construct
cttggcagggttgaggtgggggaggaggaggtagttagag

SEQ ID NO: 61 - DNA, 40 bp, Artificial Sequence
  Synthetic construct
tttgggagggttgaggtgggggaggaggaggcagttagag

SEQ ID NO: 62 - DNA, 85 bp, Artificial Sequence
  Synthetic construct
tagggaagagaaggacaatgatggtggaggttatatactatccgggtgggtttagggggggtttgactagtacatgaccacttga

SEQ ID NO: 63 - DNA, 85 bp, Artificial Sequence
  Synthetic construct
tagggaagagaaggacaatgatggggtgggttaactttttgggggggttgttattttggggattgactagtacatgaccacttga

SEQ ID NO: 64 - DNA, 85 bp, Artificial Sequence
  Synthetic construct
tagggaagagaaggacaatgatggggggaagggtgggactttaaccatacttcggtggcatgttgactagtacatgaccacttga

SEQ ID NO: 65 - DNA, 85 bp, Artificial Sequence
  Synthetic construct
tagggaagagaaggacaatgattttgggagggttgaggtgggggaggaggaggtagttagagttgactagtacatgaccacttga

SEQ ID NO: 66 - DNA, 85 bp, Artificial Sequence
  Synthetic construct
tagggaagagaaggacaatgattttgggcgggttgaggtgggggaggaggaggtagttagagttgactagtacatgaccacttga

SEQ ID NO: 67 - DNA, 85 bp, Artificial Sequence
  Synthetic construct
tagggaagagaaggacaatgatgctcgggcgggtggagggtagtttgcgcgggggtggaggtttgactagtacatgaccacttga

SEQ ID NO: 68 - DNA, 40 bp, Artificial Sequence
  Synthetic construct
ccaccacacacacacacaaccaccctacagtcctgtacga

SEQ ID NO: 69 - DNA, 40 bp, Artificial Sequence
  Synthetic construct
acaccaccacactcatccaccacacacacccgcgcctcca

SEQ ID NO: 70 - DNA, 40 bp, Artificial Sequence
  Synthetic construct
ccaccacacacacacacaaccaccctacagtcctatacga

SEQ ID NO: 71 - DNA, 38 bp, Artificial Sequence
  Synthetic construct
ccaccacacacacacaaccaccctacagtcctgtacga

SEQ ID NO: 72 - DNA, 40 bp, Artificial Sequence
  Synthetic construct
accacgccacacccgggacacacacgccacagacctctgc

SEQ ID NO: 73 - DNA, 40 bp, Artificial Sequence
  Synthetic construct
accgcacaacaacaccaaacccaacgcttttgaaccgcta

SEQ ID NO: 74 - DNA, 39 bp, Artificial Sequence
  Synthetic construct
ccccacacacccccacaacacaccactcacggctcccca

SEQ ID NO: 75 - DNA, 40 bp, Artificial Sequence
  Synthetic construct
gcacaccaccaccgcgagagcccccgacctgcacccctgc

SEQ ID NO: 76 - DNA, 40 bp, Artificial Sequence
  Synthetic construct
cacacacacccctggctccaaaccacccaacctttggcga

SEQ ID NO: 77 - DNA, 40 bp, Artificial Sequence
  Synthetic construct
caggcaacaacacaccacgcgcacacacctttacgtcaca

SEQ ID NO: 78 - DNA, 40 bp, Artificial Sequence
  Synthetic construct
caccaccacacctacgctccaattacctaccccgggcccc

SEQ ID NO: 79 - DNA, 40 bp, Artificial Sequence
  Synthetic construct
cacacacacccctggctccaaaccacccaaccttcggcga

SEQ ID NO: 80 - DNA, 40 bp, Artificial Sequence
  Synthetic construct
accacacaccacgagatttagggcgccacaccactccgtc

SEQ ID NO: 81 - DNA, 40 bp, Artificial Sequence; identical to SEQ ID NO: 24
  Synthetic construct

SEQ ID NO: 82 - DNA, 40 bp, Artificial Sequence
  Synthetic construct
caccgcaggatactacacaccagcacacagccaccacaca

SEQ ID NO: 83 - DNA, 40 bp, Artificial Sequence
  Synthetic construct
accaccacgcaccacaccaccacaccgagactgccccacg

SEQ ID NO: 84 - DNA, 40 bp, Artificial Sequence
  Synthetic construct
acacacacccacctagcgcaggacacaccagtatccaccc

SEQ ID NO: 85 - DNA, 40 bp, Artificial Sequence; identical to SEQ ID NO: 8
  Synthetic construct

SEQ ID NO: 86 - DNA, 40 bp, Artificial Sequence
  Synthetic construct
ccaacaccatacacagcacacaaccagagacgccacatga

SEQ ID NO: 87 - DNA, 40 bp, Artificial Sequence
  Synthetic construct
agcagcccaacacatccacactcccacagcccctctcctg

SEQ ID NO: 88 - DNA, 40 bp, Artificial Sequence
  Synthetic construct
gagcagccaacaacaaaacaaagaacagcacaccacagcc

SEQ ID NO: 89 - DNA, 40 bp, Artificial Sequence
  Synthetic construct
cacacgcacacacgacacaaaaccggataacacagcactg

SEQ ID NO: 90 - DNA, 40 bp, Artificial Sequence
  Synthetic construct
gccaggccacagcactacacaccaccacacaccggccaga

SEQ ID NO: 91 - DNA, 40 bp, Artificial Sequence
  Synthetic construct
cccacacccacgcgcggaaccacccacacactcacccagc

SEQ ID NO: 92 - DNA, 40 bp, Artificial Sequence
  Synthetic construct
caccacacgcaccaccctccaccccgccccagagccgccg

SEQ ID NO: 93 - DNA, 37 bp, Artificial Sequence
  Synthetic construct
cacacacacaccaacagccactgctaactctcctagg

SEQ ID NO: 94 - DNA, 40 bp, Artificial Sequence
  Synthetic construct
caccacacacatcacccacacgacgccaacacagcgcggt

SEQ ID NO: 95 - DNA, 40 bp, Artificial Sequence; identical to SEQ ID NO: 68
  Synthetic construct

SEQ ID NO: 96 - DNA, 40 bp, Artificial Sequence; identical to SEQ ID NO: 69
  Synthetic construct

SEQ ID NO: 97 - DNA, 40 bp, Artificial Sequence; identical to SEQ ID NO: 70
  Synthetic construct

SEQ ID NO: 98 - DNA, 38 bp, Artificial Sequence; identical to SEQ ID NO: 71
  Synthetic construct

SEQ ID NO: 99 - DNA, 40 bp, Artificial Sequence; identical to SEQ ID NO: 73
  Synthetic construct

SEQ ID NO: 100 - DNA, 40 bp, Artificial Sequence; identical to SEQ ID NO: 2
  Synthetic construct

SEQ ID NO: 101 - DNA, 40 bp, Artificial Sequence; identical to SEQ ID NO: 86
  Synthetic construct

SEQ ID NO: 102 - DNA, 40 bp, Artificial Sequence; identical to SEQ ID NO: 87
  Synthetic construct

SEQ ID NO: 103 - DNA, 40 bp, Artificial Sequence; identical to SEQ ID NO: 92
  Synthetic construct

SEQ ID NO: 104 - DNA, 37 bp, Artificial Sequence; identical to SEQ ID NO: 93
  Synthetic construct

SEQ ID NO: 105 - DNA, 40 bp, Artificial Sequence
  Synthetic construct
gcccaaccgatcaacacacacatacacacacattggatca

SEQ ID NO: 106 - DNA, 40 bp, Artificial Sequence
  Synthetic construct
cacacacacacacgaacaaccaacaatctcgtagcggcta

SEQ ID NO: 107 - DNA, 40 bp, Artificial Sequence
  Synthetic construct
cacacacacacacgcacatcatcatggttagtctgattca

SEQ ID NO: 108 - DNA, 41 bp, Artificial Sequence
  Synthetic construct
cggggcagaacacacacactacaggacacacacttcgttac

SEQ ID NO: 109 - DNA, 40 bp, Artificial Sequence
  Synthetic construct
cacacacacacacacaacaaagagtgctaaagacggcagc

SEQ ID NO: 110 - DNA, 40 bp, Artificial Sequence
  Synthetic construct
cacacacacggacacacacggtacaatatcacggagagtc

SEQ ID NO: 111 - DNA, 40 bp, Artificial Sequence
  Synthetic construct
gcagcaaggagtagactcaacacacacacacactaccctg

SEQ ID NO: 112 - DNA, 40 bp, Artificial Sequence
  Synthetic construct
cacacacacacacgcttggatttaacacgccaggacaatg

SEQ ID NO: 113 - DNA, 40 bp, Artificial Sequence
  Synthetic construct
cacacacatacacacacacaacttgcttattcggcgaagt

SEQ ID NO: 114 - DNA, 40 bp, Artificial Sequence
  Synthetic construct
cacacacacgcacactgacattcaaggctattacgctcta

SEQ ID NO: 115 - DNA, 40 bp, Artificial Sequence
  Synthetic construct
cacacacacacacacacagtcgggtaaactcctccatttg

SEQ ID NO: 116 - DNA, 40 bp, Artificial Sequence
  Synthetic construct
cacacacacacacgaaccatataacattgcagttgcctag

SEQ ID NO: 117 - DNA, 40 bp, Artificial Sequence; identical to SEQ ID NO: 77
  Synthetic construct

SEQ ID NO: 118 - DNA, 40 bp, Artificial Sequence
  Synthetic construct
cacacacacacacactgacagtacatcggttagtatccgg

SEQ ID NO: 119 - DNA, 40 bp, Artificial Sequence
  Synthetic construct
gaccacacacccaaaccacacacacttaacacacaccgta

SEQ ID NO: 120 - DNA, 40 bp, Artificial Sequence
  Synthetic construct
cacacacacacatgcacacccttacttagcttggcgacgt

SEQ ID NO: 121 - DNA, 40 bp, Artificial Sequence
  Synthetic construct
ggaccacgatacacacacacactatacacttgggcgatga

SEQ ID NO: 122 - DNA, 40 bp, Artificial Sequence
  Synthetic construct
cacacaacacacaccacacagttaaagatgattcggggct

SEQ ID NO: 123 - DNA, 40 bp, Artificial Sequence
  Synthetic construct
ggggggaagggtgggactttaaccatacttcggtggcatg

SEQ ID NO: 124 - DNA, 40 bp, Artificial Sequence
  Synthetic construct
caagtgcatgtcgtgcgtcgggtagattgggtgggttggg

SEQ ID NO: 125 - DNA, 40 bp, Artificial Sequence
  Synthetic construct
ggggggggtaaagagaggcgttaaagtggcgttgttggtc

SEQ ID NO: 126 - DNA, 40 bp, Artificial Sequence
  Synthetic construct
ttgaatagcgccagtcgtaatcatcgatatgggactgtaa

SEQ ID NO: 127 - DNA, 40 bp, Artificial Sequence
  Synthetic construct
cacgtgcatgtcgtgcgtcgggtagattgggtgggttggg

SEQ ID NO: 128 - DNA, 40 bp, Artificial Sequence
  Synthetic construct
gctcgggcgggtggagggtagtttgcgcgggggtggaggt

SEQ ID NO: 129 - DNA, 40 bp, Artificial Sequence
  Synthetic construct
caagtgcatgtcgtgcgtcgggtagattgggcgggttggg

SEQ ID NO: 130 - DNA, 40 bp, Artificial Sequence
  Synthetic construct
gtgggtgggatattggtggtggtgcgctaaagtgtattgg

SEQ ID NO: 131 - DNA, 40 bp, Artificial Sequence
  Synthetic construct
ggggggggtagatgcggtaggccggtaggcattcaatggt

SEQ ID NO: 132 - DNA, 40 bp, Artificial Sequence
  Synthetic construct
ttgactagcgccagtcgtaatcatcgatatgggactgtaa

SEQ ID NO: 133 - DNA, 40 bp, Artificial Sequence
  Synthetic construct
ttgaatagcgccagtcgtaatcatcgatatgggactgtac

SEQ ID NO: 134 - DNA, 40 bp, Artificial Sequence
  Synthetic construct
ttgaatagagccagtcgtaatcatcgatatgggactgtaa

SEQ ID NO: 135 - DNA, 40 bp, Artificial Sequence
  Synthetic construct
ttgaatagcgacagtcgtaatcatcgatatgggaatgtaa

SEQ ID NO: 136 - DNA, 40 bp, Artificial Sequence
  Synthetic construct
ttgaatagcgacagtcgtaatcatcgatatgggactgtaa

SEQ ID NO: 137 - DNA, 40 bp, Artificial Sequence
  Synthetic construct
ttgaacagcgccagtcgtaatcatcgatatgggactgtaa

SEQ ID NO: 138 - DNA, 40 bp, Artificial Sequence
  Synthetic construct
ttgaatagcgccagtcgtaatcatcgatatgggaatgtaa

SEQ ID NO: 139 - DNA, 40 bp, Artificial Sequence
  Synthetic construct
tttaatagcgccagtcgtaatcatcgatatgggactgtaa

SEQ ID NO: 140 - DNA, 40 bp, Artificial Sequence
  Synthetic construct
ttgaatagcgccagtcgcaatcatcgatatgggactgtaa

SEQ ID NO: 141 - DNA, 40 bp, Artificial Sequence
  Synthetic construct
ttgagtagcgccagtcgtaatcatcgatatgggactgtaa

SEQ ID NO: 142 - DNA, 40 bp, Artificial Sequence
  Synthetic construct
ctgaatagcgccagtcgtaatcatcgatatgggactgtaa

SEQ ID NO: 143 - DNA, 40 bp, Artificial Sequence
  Synthetic construct
ttgactagcgacagtcgtaatcatcgatatgggactgtaa

SEQ ID NO: 144 - DNA, 40 bp, Artificial Sequence
  Synthetic construct
ttgaatagcgccagtcgtaatcatcgatatgggagtgtaa

SEQ ID NO: 145 - DNA, 40 bp, Artificial Sequence
  Synthetic construct
ttgactagagccagtcgtaatcatcgatatgggactgtaa

SEQ ID NO: 146 - DNA, 40 bp, Artificial Sequence
  Synthetic construct
ttgaatagggccagtcgtaatcatcgatatgggactgtaa

SEQ ID NO: 147 - DNA, 40 bp, Artificial Sequence
  Synthetic construct
ttgaatagtgccagtcgtaatcatcgatatgggactgtaa

SEQ ID NO: 148 - DNA, 40 bp, Artificial Sequence
  Synthetic construct
ttgaatagcgtcagtcgtaatcatcgatatgggactgtaa

SEQ ID NO: 149 - DNA, 40 bp, Artificial Sequence
  Synthetic construct
ttgaatagcgccagtcgtaatcatcgatataggactgtaa

SEQ ID NO: 150 - DNA, 40 bp, Artificial Sequence
  Synthetic construct
gctaggcttacgtagggaactcgtaccgtggttagcaatc

SEQ ID NO: 151 - DNA, 40 bp, Artificial Sequence
  Synthetic construct
ttgaatagcgccagtcgtaatcatcgctatgggactgtaa

SEQ ID NO: 152 - DNA, 40 bp, Artificial Sequence
  Synthetic construct
ttgaatagcgccagtcgtaatcatcgatatgcgactgtaa

SEQ ID NO: 153 - DNA, 40 bp, Artificial Sequence
  Synthetic construct
ctcgactcgtacgccaacttaaactgagttgttgcgcccc

SEQ ID NO: 154 - DNA, 40 bp, Artificial Sequence
  Synthetic construct
catggcgggtgggggggcagaaggggggggggggggtttt

SEQ ID NO: 155 - DNA, 40 bp, Artificial Sequence
  Synthetic construct
gtggggggggttgcaaaggttagaggtgtagggaggtggt

SEQ ID NO: 156 - DNA, 40 bp, Artificial Sequence
  Synthetic construct
tttgggagggtagaggtgggggaggcggaggtagttagag

SEQ ID NO: 157 - DNA, 40 bp, Artificial Sequence
  Synthetic construct
tttgggagggtggaggtgggggaggaggaggtagttagag

SEQ ID NO: 158 - DNA, 40 bp, Artificial Sequence
  Synthetic construct
tttgggagggttgaggggggggaggaggaggtagttagag

SEQ ID NO: 159 - DNA, 22 bp, Artificial Sequence
  Synthetic construct
tagggaagagaaggacaatgat

SEQ ID NO: 160 - DNA, 23 bp, Artificial Sequence
  Synthetic construct
ttgactagtacatgaccacttga

SEQ ID NO: 161 - DNA, 23 bp, Artificial Sequence
  Synthetic construct
tagggaagagaaggacatatgat

SEQ ID NO: 162 - DNA, 23 bp, Artificial Sequence
  Synthetic construct
ttgactagtacatgaccacttga

SEQ ID NO: 163 - DNA, 41 bp, Artificial Sequence
  Synthetic construct
ttttttagggaagagaaggacaatgattttgggcgggttga

SEQ ID NO: 164 - DNA, 41 bp, Artificial Sequence
  Synthetic construct
aaaaatagggaagagaaggacaatgattttgggcgggttga

SEQ ID NO: 165 - DNA, 85 bp, Artificial Sequence
  Synthetic construct
tagggaagagaaggacaatgattttgggagggttgaggcgggggaggaggaggtagttagagttgactagtacatgaccacttga

SEQ ID NO: 166 - DNA, 85 bp, Artificial Sequence
  Synthetic construct
tagggaagagaaggacaatgattacgggtggaggggggggcggttggttgtagttatttggtttgactagtacatgaccacttga

SEQ ID NO: 167 - DNA, 85 bp, Artificial Sequence
  Synthetic construct
tagggaagagaaggacaatgattttgggggggttgaggtgggggaggaggaggtagttagagttgactagtacatgaccacttga

SEQ ID NO: 168 - DNA, 85 bp, Artificial Sequence
  Synthetic construct
tagggaagagaaggacaatgatcaagtgcatgtcgtgcgtcgggtagattgggcgggttgggttgactagtacatgaccacttga

SEQ ID NO: 169 - DNA, 85 bp, Artificial Sequence
  Synthetic construct
tagggaagagaaggacaatgatcatggcggggggggggggagaaggggggggggggggttttttgactagtacatgaccacttga

SEQ ID NO: 170 - DNA, 85 bp, Artificial Sequence; identical to SEQ ID NO: 67
  Synthetic construct

(19) United States
(12) Patent Application Publication
(10) Pub. No.: US 2024/0287527 A1
(43) Pub. Date: Aug. 29, 2024

(54) VIRAL PROTEIN SPECIFIC APTAMERS AND METHODS OF USE IN DIAGNOSTICS, THERAPEUTIC PURPOSES

(71) Applicant: AYASS BIOSCIENCE LLC, Frisco, TX (US)

(72) Inventors: Mohammad Ammar AYASS, San Angelo, TX (US); Lina ABI MOSLEH, Frisco, TX (US)

(21) Appl. No.: 18/572,430

(22) PCT Filed: Jun. 30, 2022
(86) PCT No.: PCT/US2022/035718

§ 371 Date: Dec. 20, 2023

Related U.S. Application Data
(60) Provisional application No. 63/216,630, filed on Jun. 30, 2021.

(51) Int. Cl. C12N 15/115; G01N 33/53; G01N 33/569

(52) U.S. Cl. CPC C12N 15/115; G01N 33/5308; G01N 33/56983; C12N 2310/16; C12N 2310/3517; G01N 2333/165

(57) ABSTRACT

Disclosed are novel aptamers that inhibit the binding of a coronaviral S protein to ACE-2 Receptor and methods of their use.

This application claims the benefit of U.S. Provisional Application No. 63/216,630, filed on Jun. 30, 2021, which is incorporated herein by reference in its entirety.

### I. BACKGROUND

Current diagnostic test products and treatments for Coronaviral infection are limited in terms of speed, stability, specificity, reliability, storage and longevity requirements and costs. The closest devices and/or methods are: (1) the antibody based tests for—either ELISA or turbidimetric; (2) laborious PCR based test; and (3) the Latex-based qualitative and semi-quantitative tests. All of the existing diagnostic tests for detecting coronavirus employ a methodologies that are less stable, more expensive, and mostly not point of-care capable. What is needed is a new set of diagnostic tests that do not suffer from these deficiencies.

### II. SUMMARY

Herein is described a number of DNA/RNA sequences (aptamers) that selectively bind viral proteins (for example, a coronavirus S protein) and are demonstrated to have use for numerous clinical and research diagnostic and therapeutic targeted technologies.

In one aspect, disclosed herein are isolated nucleic acid comprising sequence as set forth in any of SEQ ID NOs: 1-158 and/or 162-170 (including, but not limited to the RNA equivalent of any nucleic acid set forth in SEQ ID NOs: 1-158 and/or 162-170), or any fragment or variant thereof comprising at least 87% sequence identity thereto. In some aspects, the nucleic acid binds to the S protein of a coronavirus (such as, for example, binding to the S protein that inhibits binding of the S protein to the ACE2 receptor). In some aspects, the nucleic acid can further comprise a detectable tag (such as, for example, a latex bead, magnetic bead, fluorescence label; fluorescent probe, chemiluminescent labels, radiolabels, and/or nanoparticle probe).

Also disclosed herein are compositions comprising one or more of the isolated nucleic acids of any preceding aspect. In some aspects, the composition can further comprise a nanoparticle or hydrogel, wherein the isolated nucleic acid is contained within the nanoparticle or hydrogel.

In one aspect, disclosed herein are kits comprising one or more of the nucleic acids of any preceding aspect.

Also disclosed herein are methods of detecting a viral infection in a subject comprising obtaining a biologic sample from the subject and measuring the concentration of a viral protein (such as, for example a coronavirus S protein) in the subject using one or more of the nucleic acids, compositions, or kits of any preceding aspect. For example, disclosed herein are methods of detecting a viral infection in a subject comprising obtaining a biologic sample from the subject and measuring the concentration of a viral protein in the subject using one or more of the nucleic acids, compositions, or kits of any preceding aspect, wherein the viral infection is selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, A246-252, L452Q, F490S, D614G, and T859N mutations in C.37 (Lambda), SARS-CoV-2 with T478K, Q498R, and H655Y mutations in B.1.1.529 (Omicron)), or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In one aspect, disclosed herein are methods of treating, inhibiting, reducing, decreasing, ameliorating, and/or preventing a viral infection in a subject comprising administering to the subject one or more of the nucleic acids or compositions of any preceding aspect. For example, disclosed herein are methods of treating, inhibiting, reducing, decreasing, ameliorating, and/or preventing a viral infection in a subject comprising administering to the subject one or more of the nucleic acids or compositions of any preceding aspect, wherein the viral infection is selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, A246-252, L452Q, F490S, D614G, and T859N mutations in C.37 (Lambda), SARS-CoV-2 with T478K, Q498R, and H655Y mutations in B.1.1.529 (Omicron)), or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2. In some aspects, the nucleic acid binds to the S protein of a coronavirus (such as, for example, binding to the S protein that inhibits binding of the S protein to the ACE2 receptor).

In some aspect, the treatment can involve the administration of a combination of two or more nucleic acids from SEQ ID NOs: 12-158 and/or 162-170 either separately or in combination in a single composition. Thus, in one aspect, disclosed herein are methods of treating, inhibiting, reducing, decreasing, ameliorating, and/or preventing a viral infection in a subject, wherein the one or more nucleic acids are selected from the group consisting of a) S10, S1I, SB1L (also referred to herein as AYA2012001 L), SB3L (also referred to herein as AYA2012004 L), SB5L (also referred to herein as AYA2012009 L), RR68, RR74, RR80; b) S9, S11, SB7, SB8 (also referred to herein as AYA2012003), SB11 (also referred to herein as AYA2012006), S1, SB16; c) S10, RR74, S2L; d) SIL, S2L, S11, RR80, RR83; and/or e) S4, S5, S12, SB6 (also referred to herein as AYA2012002), RR84, RR87, RR92, RR93.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A and 1B show an illustration of the SELEX procedure for the development of SARS-CoV-2 trimer S protein specific neutralizing aptamers. FIG. 1A shows a traditional SELEX procedure. A library of single-stranded DNA oligonucleotides (10¹⁵ different unique sequences) was used. Each unique sequence contains random bases (40 nt) flanked by two primer binding sites, which are used for PCR amplification. In the selection step, the library was incubated with SARS-CoV-2 trimer S protein immobilized on Ni-NTA magnetic beads, the unbound sequences were separated from those that bound, and target-bound sequences were eluted from target molecules and amplified by PCR using biotinylated reverse primers. PCR product was pulled down using streptavidin beads and the specific single stranded DNA was separated from complementary biotinylated strand with sodium hydroxide solution and utilized for the next round of selection. FIG. 1B shows the SELEX procedure for neutralizing aptamers selection. His-tagged trimer SARS-CoV-2 S protein was pre-incubated with Ni-NTA beads. Human ACE2 receptors was added to SARS-CoV-2 trimer S protein immobilized on the beads to block the receptor binding site on S protein. The eluted ssDNA from round 6 of selection was incubated with the protein complex (S protein plus ACE2 receptor) immobilized on the beads. The flowthrough, representing aptamers that specifically bind to trimer S protein site concealed by ACE2 receptors were collected and proceeded for 6 more rounds of traditional selection. After round 12 of selection, ssDNA was sequenced.

FIGS. 2A, 2B, and 2C show aptamers binding to recombinant SARS-CoV-2 trimer S protein. FIG. 2A shows an ELISA based binding assay of the most enriched aptamer sequences after 12 rounds of selection. SARS-CoV-2 trimer S protein was immobilized on MaxiSorp plate and 10 nM of biotinylated aptamers were incubated with the immobilized protein. Bound aptamers were detected using Streptavidin-HRP. FIG. 2B shows an ELISA based competition assay for the binding of the aptamers to SARS-CoV-2 trimer S protein. SARS-CoV-2 trimer S protein was immobilized on MaxiSorp plate and 10 nM biotinylated aptamers were added in the absence or presence of 100-fold excess of non-biotinylated respective aptamers. Bound aptamers were detected using Streptavidin-HRP. Each value was the average of duplicate measurements. Error bars represent mean±SD(σ). FIG. 2C shows the predicted secondary structure of selected aptamers that bind to SARS-CoV-2 S protein. The predicted secondary structures of (a) AYA2012001 (also referred to herein as SB1 (SEQ ID NO: 35), (b) AYA2012002 (also referred to herein as SB6) (SEQ ID NO: 37), (c) AYA2012003 (also referred to herein as SB8) (SEQ ID NO: 39), (d) AYA2012004 (also referred to herein as SB3) (SEQ ID NO: 36), (e) AYA2012006 (also referred to herein as SB11) (SEQ ID NO: 40), (f) AYA2012007 (also referred to herein as SB9) (SEQ ID NO: 129), (g) AYA2012008 (also referred to herein as SB7) (SEQ ID NO: 38), (h) AYA2012009 (also referred to herein as SB5) (SEQ ID NO: 128). The secondary structures were obtained using the mFold webserver.

FIGS. 3A and 3B Aptamers' inhibition of the binding of SARS-CoV-2 trimer S protein to ACE2 receptors expressed on the surface of Vero E6 cells. FIG. 3A shows inhibition of binding of SARS-CoV-2 trimer S protein to ACE2 receptors expressed on the surface of Vero E6 cells. S protein at 10 nM was added to Vero E6 cells after preincubation with 5 μM of indicated aptamer. Random 40 nucleotides ssDNA was used as a control. After 1 hr incubation, unbound S protein was washed out and the cell pellet was solubilized with 1% Triton X100. Supernatant containing solubilized His-tagged SARS-CoV-2 trimer S protein was applied to Nickel Coated plate. His-tagged S protein bound to Nickel was detected using mouse monoclonal antibodies against S protein and Horseradish Peroxidase-conjugated goat anti-mouse antibodies. Each value was the average of duplicate measurements. Error bars represent mean±SD(σ). P-value were determined with one-way Anova, Dunnett's multiple comparison test. **** denotes p<0.0001. FIG. 3B shows binding affinity for aptamers with the best inhibitory activity. Characterization of the affinity between aptamers AYA2012001, AYA2012004, AYA2012006 and SARS-CoV-2 trimer S protein with surface plasmon resonance (SPR). Random 40 nucleotide ssDNA was introduced as control.

FIGS. 4A, 4B, 4C, 4D, 4E, and 4F show ELISA based binding assay of selected aptamers to variants of SARS-CoV-2 trimer S protein. Recombinant SARS-CoV-2 trimer S protein from (4A) Alpha, (4B) Lambda, (4C) Mu, (4D) Delta, (4E) Delta plus, and (4F) Omicron variants were immobilized on MaxiSorp plate. 10 nM of Biotinylated aptamers were incubated with indicated variant for 1 hr. Bound aptamers were detected using Streptavidin-HRP. Each value was the average of duplicate measurements. Error bars represent mean±SD(σ).

FIGS. 5A and 5B show a characterization of selected aptamers containing Primer Sequences (long aptamers). FIG. 5A shows an ELISA based binding assay of the selected aptamers containing primer sequences (long aptamers). SARS-CoV-2 trimer S protein was immobilized on MaxiSorp plate and biotinylated aptamers were added at 10 nM concentration for 1 hr incubation with the protein. Bound aptamers were detected using Streptavidin-HRP. FIG. 5B shows cell based binding assay of SARS-CoV-2 trimer S protein to ACE2 expressing Vero E6 cells in the absence or presence of the selected aptamers containing primer sequences (long aptamers). His-tagged SARS-CoV-2 trimer S protein at 10 nM was added to Vero E6 cells after preincubation with 5 μM of indicated aptamer. After 1 hr incubation, unbound trimer S protein was washed out and the cell pellet was solubilized with 1% Triton X100. Supernatant containing solubilized trimer S protein was applied to Nickel Coated plate. His-tagged trimer S protein bound to Nickel was detected using mouse monoclonal antibodies against S protein and Horseradish Peroxidase-conjugated goat anti-mouse antibodies. Each value was the average of duplicate measurements. Error bars represent mean±SD(σ). P-value were determined with one-way Anova, Dunnett's multiple comparison test. **** denotes p<0.0001.

FIGS. 6A and 6B show characterization of modified aptamers. Figure A shows aptamers modification. The predicted secondary structures of (a) AYA2012004 L (also referred to herein as SB3L (SEQ ID NO: 66), (b) its truncated modification AYA2012004 L-M1 (SEQ ID NO: 162) and (c) the duplex AYA2012004 L-M2 (SEQ ID NOs: 163 and 164). The secondary structure analysis of AYA2012004 L was performed using the mfold webserver. The default environment was used for folding, which includes 37° C. as the folding temperature and 0.2 M of Na+. The secondary structure of AYA2012004 L as well as its truncated modification AYA2012004 L-M1 (5′-TAGGGAAGAGAAGGACAATGATTTTGGGCGGGTTGA-3′)(SEQ ID NO: 162) are shown. The truncation was performed by removing the big loop (nucleotides from position 38 to 85) from the AYA2012004 L. The AYA2012004 L-M2 was made by adding a TTTTT and AAAAA primes to AYA2012004 L-M1's 5-end (i.e., 5′-TTTTTTAGGGAAGAGAAGGACAATGATTTTGGGCGGGTTGA-3′)(SEQ ID NO: 163) and AAAAATAGGGAAGAGAAGGACAATGATTTTGGGCGGGTTGA-3′)(SEQ ID NO: 164), such that two copies of modified AYA2012004 L-M1 will have a tendency to combine together, forming AYA2012004 L-M2 and potentially cover more interaction surface of the S protein. FIG. 6B shows cell based binding assay of SARS-CoV-2 S-protein trimer to ACE2 expressing Vero E6 cells in the absence or presence of the modified aptamers. His-tagged SARS-CoV-2 trimer S protein (10 nM) was incubated with Vero E6 cells in the absence or presence of the indicated aptamers (5 M). After 1 hr incubation, unbound S-protein was washed out and the cell pellet was solubilized with 1% Triton X100 for 1 hr. Supernatant containing solubilized S protein was applied to Nickel Coated plate. His-tagged trimer S protein bound to Nickel was detected using mouse monoclonal antibodies against S protein. Each value was the average of duplicate measurements. Error bars represent mean±SD(σ). P-value were determined with one-way Anova, Dunnett's multiple comparison test. **** denotes p<0.0001.

FIG. 7 shows binding and specificity determination of modified aptamers to different variants of S protein with Surface Plasmon Resonance(SPR). Characterization of the binding affinity between selected aptamers and SARS-CoV-2 trimer S protein Wuhan original strain, Alpha, and Delta variants with Surface Plasmon Resonance (SPR). The affinity of selected aptamers were determined via SPR. Kd values are presented in the table.

FIGS. 8A and 8B show flow cytometry study on inhibition of SARS-CoV-2 trimer S protein binding to ACE2 receptors on the surface of Vero E6 cells by selected aptamers. FIG. 8A shows concentration dependent inhibition of SARS-CoV-2 trimer S protein binding to Vero E6 cells by aptamer AYA2012004 L. SARS-CoV-2 trimer S protein was pre-incubated for 1 hr in absence or presence of aptamer (AYA2012004 L) at different concentrations, anti-SARS-CoV-2 RBD neutralizing antibodies or control aptamer with random 40 nucleotides ssDNA in 100 ul PBS supplemented with 1 mM Mg2+ as indicated. S protein with or without aptamers or anti-SARS-CoV-2 RBD neutralizing antibodies were then incubated for 30 min with Vero E6 cells (10⁵ per well) at 4° C. followed by washing two times. Cells were stained with secondary antibody, anti-his-tag-APC or control antibody and fixable viability dye eFluor 450 on 4° C. for 30 min, followed by washing. Cells were fixed and acquired by flow cytometry. Anti-SARS-CoV-2 RBD neutralizing antibody (50 nM) was used as a positive control for aptamer inhibition and BSA or none was used as negative control for trimer S protein binding. Each value was the average of triplicate measurements. P-value were determined with two-way Anova, Turkey's multiple comparison test. ** denotes p<0.002, *** denotes p<0.0002, and **** denotes p<0.0001. FIG. 8B shows inhibition of different SARS-CoV-2 trimer S-protein variants binding to ACE2 receptors on the surface of Vero E6 cells by selected aptamers. Trimer S protein (12.5 nM) from the indicated SARS-CoV-2 strains was pre-incubated in the absence or presence of 5 M of aptamers (AYA2012004 L, AYA2012004 L-M1 and AYA2012004 L-M2) in 100 ul PBS plus 1 mM MgCl2 for 1 hr at room temperature, followed by incubation with Vero E6 cells (10⁵ per well) for 30 min at 4° C. After incubation cells were washed two times and stained with secondary antibody, anti-his-tag-APC or control antibody with fixable viability dye eFluor 450 on 4° C. for 30 min, followed by washing, fixing and then acquired by flow cytometry. Anti-SARS-CoV-2 RBD neutralizing antibody (30 nM) was used as a positive control for aptamers inhibition. Data from one representative of three independent experiments are shown. Results are shown in percent of binding of S protein. Error bars represent mean±SD(σ). P-value were determined with one-way Anova, Dunnett's multiple comparison test. **** denotes p<0.0001.

FIG. 9 shows inhibition binding of Omicron SARS-CoV-2 trimer S protein to ACE2 receptors on the surface of Vero E6 cells by selected aptamers. His-tagged Omicron SARS-CoV-2 trimer S protein was incubated with Vero E6 cells in the absence or presence of the indicated aptamers or anti-SARS-CoV-2 RBD neutralizing Ab at 30 nM. After 1 hr incubation, unbound S protein was washed out and the cell pellet was solubilized with 1% Triton X100 for 1 hr. Supernatant containing solubilized S protein was applied to Nickel Coated plate. His-tagged trimer S protein bound to Nickel was detected using mouse monoclonal antibodies against S protein and Horseradish Peroxidase-conjugated goat anti-mouse antibodies. Each value was the average of duplicate measurements. Error bars represent mean±SD(σ). P-value were determined with one-way Anova, Dunnett's multiple comparison test. * denotes p<0.015 and **** denotes p<0.0001.

FIG. 10 shows blocking of the SARS-CoV-2 trimer S protein VLPs uptake to ACE2 receptors of transfected HEK293T cells by aptamers. VLPs were preincubated for 30 min with or without aptamers (5 uM) in PBS plus 1.0 mM MgCl2 at room temperature as indicated. Pre-incubated VLPs of each condition were then added to mock or ACE2 receptors overexpress HEK293T cells and incubated at 37° C. in a humidified 5% CO2 atmosphere. After 72 hr, cells were washed two times with 1×PBS, then stained with fixable viability dye eFluor 450 at 4° C. for 30 min. Binding and subsequent uptake of VLPs to ACE2 receptors transfected HEK293T cell in the presence or absence of aptamers was determined by flow cytometry. Data from one representative of three independent experiments is shown. Results are shown in percent of GFP signal of VLPs to HEK293T ACE2 cells. Error bars represent mean±SD (a). P-value were determined with one-way Anova, Dunnett's multiple comparison test. * denotes p<0.05 and ** denotes p<0.001.

FIGS. 11A, 11B, 11C, 11D, 11E, 11F, 11G, and 11H show predicted secondary structures of (11a) AYA2012001_L (SEQ ID NO: 65), (11b) AYA2012002_L (SEQ ID NO: 165), (11c) AYA2012003_L (SEQ ID NO: 166), (11d) AYA2012004_L (SEQ ID NO: 65), (11e) AYA2012006_L (SEQ ID NO: 167), (11f) AYA2012007_L (SEQ ID NO: 168), (11g) AYA2012008_L (SEQ ID NO: 169), (11h) 1AYA2012009_L (SEQ ID NO: 170).

FIG. 12 shows concentration dependence inhibition of SARS-CoV-2 trimer S protein binding to Vero E6 cells by selected aptamers. His-tagged SARS-CoV-2 trimer S protein was incubated with Vero E6 cells in the absence or presence of the indicated aptamers at different concentrations. After 1 hr incubation, unbound S-protein was washed out and the cell pellet was solubilized with 1% Triton X100 for 1 hr. Supernatant containing solubilized S protein was applied to Nickel Coated plate. His-tagged trimer S protein bound to Nickel was detected using mouse monoclonal antibodies against S protein and Horseradish Peroxidase-conjugated goat anti-mouse antibodies. Absorbance was measured at 450 nm after incubation with 3,3′,5,5′-tetramethylbenzidine (TMB) substrate.

FIG. 13 shows specificity assessment of selected aptamers using SPRi. Influenza A H1N1 hemagglutinin protein, SARS-CoV-2 Nucleocapsid protein, Human ACE2/ACEH protein and serum from healthy human donor were used to investigate specificity of selected aptamers. None of those control proteins showed detectable binding to the aptamers.

FIG. 14 shows flow cytometry based binding assay of SARS-COV-2 trimer S protein to Vero E6 cells. SARS-CoV-2 trimer S protein was incubated in absence or presence of neutralizing antibody in 100 uL of PBS supplemented with 1 mM MgCl2 as indicated at 40 C for 1 hr, followed by washing two times with PBS. Cells were then stained with secondary antibody, anti-his-tag-APC or control antibody with fixable viability dye eFluor 450 for 30 min at 40 C, followed by washing and fixing. S protein binding (% binding as indicated in gates and also on MFI shift) was determined by flow cytometry. Index of geometric mean fluorescence intensity (gMFI) (S protein staining by anti-his-tag divided by control staining).

FIGS. 15A and 15B show ACE2 receptor expression on the surface of transfected HEK293T cells. FIG. 15A shows mock transfected or ACE2 receptor transfected HEK293T cells were stained with anti-human ACE-2-APC or control antibody with fixable viability dye eFluor 450 for 30 min at 40 C, followed by washing and then fixation. ACE2 expression on MFI shift was determined by flow cytometry. FIG. 15B shows binding of SARS-CoV-2 trimer spike protein to transfected HEK 293T cells. Mock transfected or ACE2 receptor transfected HEK293T cells were incubated with or without trimer S protein (12.5 nM) in 100 uL PBS containing 1 mM MgCl2 for 30 min at 40 C, followed by washing two times with PBS. Cells were then stained with secondary antibody, anti-his-tag-APC or control antibody with fixable viability dye eFluor 450 for 30 min at 40 C, followed by washing and then fixation. S-protein binding on MFI shift was determined by flow cytometry. Index of geometric mean fluorescence intensity (gMFI) (S protein staining by anti-his-tag divided by control staining).

FIGS. 16A and 16B show predicted structure of AYA2012004_L/original strain S protein Complex. FIG. 16A shows predicted docked complex of AYA2012004_L and original strain spike protein trimer. FIG. 16B shows the zoomed view of the highlighted regions in (a). The polar interactions are represented in red dash lines, the polar residues are shown as sticks and labeled in red using three letter abbreviations for amino acids, and in black using single letter abbreviation for nucleic acids.

FIGS. 17A and 17B show the predicted structure of AYA2012004_L/Alpha-variant S protein Complex FIG. 17A shows the predicted docked complex of AYA2012004_L and the Alpha-variant spike protein trimer. FIG. 17B shows the zoomed view of the highlighted regions in (a). The polar interactions are represented in red dash lines, the polar residues are shown as sticks and labeled in red using three letter abbreviations for amino acids, and in black using single letter abbreviation for nucleic acids.

FIGS. 18A and 18B show the predicted structure of AYA2012004_L/Delta-variant S protein Complex. FIG. 18A shows the predicted docked complex of AYA2012004_L and Delta-variant spike protein trimer. FIG. 18B shows the zoomed view of the highlighted regions in (18A). The polar interactions are represented in red dash lines, the polar residues are shown as sticks and labeled in red using three letter abbreviations for amino acids, and in black using single letter abbreviation for nucleic acids.

FIGS. 19A and 19B show predicted structure of AYA2012004_L/Lambda-variant S protein Complex. FIG. 19A shows predicted docked complex of AYA2012004_L and the Lambda-variant spike protein trimer. FIG. 19B shows the zoomed view of the highlighted regions in (19A). The polar interactions are represented in red dash lines, the polar residues are shown as sticks and labeled in red using three letter abbreviations for amino acids, and in black using single letter abbreviation for nucleic acids.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular S-protein aptamer (such as, for example, any of SEQ ID Nos 1-158 and/or 162-170) is disclosed and discussed and a number of modifications that can be made to a number of molecules including the S-protein aptamer (such as, for example, any of SEQ ID Nos 1-158 and/or 162-170) are discussed, specifically contemplated is each and every combination and permutation of S-protein aptamer (such as, for example, any of SEQ ID Nos 1-158 and/or 162-170) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Prior to the present disclosure, commercially available clinical Coronaviral infections were either based on antibody technology or a PCR based assay. The ELISA method requires a primary antibody (Ab) designed to bind to the target protein, and a secondary antibody (to bind to the primary antibody) that usually carries a signal generator in the form of an enzymatic amplification platform (e.g., horseradish peroxidase) or a fluorescent label (e.g., small molecule dye or nanoparticle). In both major platforms, the expense to produce an inherently delicate antibody is the heart of the diagnostic system. Ab-based assay systems have to be stored with stabilizers in solution at a certain temperature (between 2-8° C.) and have limited shelf-life. The Ab-based reagents is often the most prohibitive in the in vitro diagnostics cost breakdown. To remedy the problems with previously existing Coronaviral detection platforms, disclosed herein are, in one aspect, are nucleic acid (DNA/RNA) or peptide sequences (i.e., aptamers) that selectively bind Coronaviral S protein (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, A246-252, L452Q, F490S, D614G, and T859N mutations in C.37 (Lambda), SARS-CoV-2 with T478K, Q498R, and H655Y mutations in B.1.1.529 (Omicron)), or MERS-CoV) and are demonstrated to have use for numerous clinical and research diagnostic and therapeutic targeted technologies.

Aptamers are molecules that interact with a target molecule (such as, for example S protein), preferably in a specific way. Typically, aptamers are small nucleic acids ranging from 15-50 bases in length (or peptides of 5-17 amino acids in length) that fold into defined secondary and tertiary structures, such as stem-loops or G-quartets. Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000-fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000-fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Accordingly, disclosed herein are isolated nucleic acid comprising sequence as set forth in any of SEQ ID NOs: 1-158 and/or 162-170 (including, but not limited to the RNA equivalent of any nucleic acid set forth in SEQ ID NOs: 1-158 and/or 162-170 and as disclosed in Tables 1, 2 and 3), or any fragment or variant thereof comprising at least 87% sequence identity thereto. In some aspects, the nucleic acid binds to the S protein of a coronavirus (such as, for example, binding to the S protein that inhibits binding of the S protein to the ACE2 receptor). In some aspects, the nucleic acid can further comprise a detectable tag (such as, for example, a latex bead, magnetic bead, fluorescence label; fluorescent probe, chemiluminescent labels, radiolabels, and/or nanoparticle probe) or any fragment, derivative, or variant thereof comprising at least 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto. In some aspects, the aptamer can comprise 5′ end and 3′ end primers TAGGGAAGAGAAGGACAATGAT (SEQ ID NO: 159) and TTGACTAGTACATGACCACTTGA (SEQ ID NO: 160); primer sequences which are used for amplification during SELEX and thus follow the formula TAGGGAAGAGAAGGACAATGAT (SEQ ID NO: 159) N40TTGACTAGTACATGACCACTTGA (SEQ ID NO: 160), where N40 is the aptamer sequence sandwiched between the 5′ and 3′ end primers.

TABLE 1
Characterized Aptamers

| | | | KD as | | Inhibition of | |
|---|---|---|---|---|---|---|
| | | | Determined | KD as | binding of | |
| | | | by ELISA | De- | Purified S | |
| SEQ | | | Based | termined | protein to ACE-2 | |
| ID | | | Binding | by SPRI | Expressed on Vero | |
| NO: | Name | Sequence | Assay (nM) | (nM) | E6 cells (%) | |
| 1 | 1G | ACACACACACAACGAACGGCAGTTACTCCGGCCCCCCTGCA | 20-100 | | | |
| 2 | 1I | ACACCCCCACCACCCTTGCACACATCACACTCCACACGCA | 20-100 | | | |
| 3 | 2A | ACCACCACACCACACCCGACCCTCCTCCTACCATCCCACC | 20-100 | | | |
| 4 | 2G | ACACGCCACACACAACGAAACTAACGCCGGGATACGCCAC | 20-100 | | | |
| 5 | 3F | GCACACACCAAACATTAGACACACAAGAACAGGGCGGCCC | 20-100 | | | |
| 6 | 4D | ACGCCACCACACACACAGCCTACCACCACGCGGAAGCTCC | 20-100 | | | |
| 7 | 4J | GCACAACCAACAACAGACAACCCACCCACCCACGCCGTTC | 20-100 | | | |
| 8 | 8 | GCACACCACACAACCAAAAGCAAGGAACCAATACCCACCG | 20-100 | | | |
| 9 | 11 | GCACACACACACATTACACAGTGATAGACCACCCAAATCC | 20-100 | | | |
| 10 | 24 | GCGCCACACACACATACACACACTAGCAGAAACACTTCGC | 20-100 | | | |
| 11 | 25 | CACACACACACACACTTCTTCGGGGGAACCAGTACGCGGT | 20-100 | | | |
| 12 | SB12 | GGGGGGAAGGGTGGGACTTTAACCATACTTCGGTGGCCTG | 20-100 | | | |
| 13 | SB13 | GGGGGGCAGGGTGGGACTTTAACCATACTTCGGTGGCATG | 20-100 | | | |
| 14 | SB14 | GGGGGGAAGGGTGGGACTTTAACCATACTTCGGTGGAATG | 20-100 | | | |
| 15 | SB15 | GGGGGGAAGGGCGGGACTTTAACCATACTTCGGTGGCATG | 20-100 | | | |
| 16 | RR70 | TTTGGGAGGGTTGAGGTGGGGGAGGTGGAGGTAGTTAGAG | 20-100 | | | |
| 17 | 1D | ACACACACCACGCACACCCACACTCTGGCCGGGACGACAG | 20-150 | | | |
| 18 | 2J | ACCACAACCCCACGTCACACTGGCTACACACCAACGCTCC | 20-150 | | | |
| 19 | 3A | ACACCACAGCCGCAAACACACACGCACAACTCCGATCGTC | 20-150 | | | |
| 20 | 3C | CACCGCAGGATACTACACACCAGCACACAGCCACTACACA | 20-150 | | | |
| 21 | 4E | GCACACACACACATTACACAGTGATAGACCACCCAAATCC | 20-150 | | | |
| 22 | 4H | ACCACCAACCACGAGGCACCTCCACCACAGCGCTTCCACC | 20-150 | | | |
| 23 | 7 | GCACACACCAAACATTAGACACACAAGAACAGGGCGGCCC | 20-150 | | | |
| 24 | 28 | CAACAAGCACACACTCACTACACACTCACACACGCCTTGG | 20-150 | | | |
| 25 | 32 | GACCACGGTACAGACAGACACACACACAGACGTTTCCCAC | 20-150 | | | |
| 26 | S9 | GGTGGAGGGTGGGAAGAGGGATGAGGTGTGTAAGGGGGGG | 20-50 | 21 | 17 | |
| 27 | S11 | GGTGGAGGTTATATACTATCCGGGCGGGTTTAGGGGGGGT | 20-50 | 100 | | |
| 28 | S12 | GGGGTTGGGGGAGGTTATGACTTTTTGGGGGGGGGACTTC | 20-50 | 20 | 14 | |
| 29 | S1 | GGTGGAGGTTATATACTATCCGGGTGGGTTTAGGGGGGGT | 20-70 | 100 | 30-50 | |
| 30 | S2 | GGGGTGGGTTAACTTTTTGGGGGGGTTGTTATTTTGGGGA | 20-70 | | 30-50 | |
| 31 | S4 | GCTAGGGCGGGTGGAGGGTAGTTTGCGCGGGGGTGGAGGT | 20-70 | 13 | 30-50 | |
| 32 | S5 | GGGGGGGTAGTAACTGTGGGTGGGGGAGGTGGAGGTTATA | 20-70 | | 30-50 | |
| 33 | S8 | GGGGGAGGGGGAGAGGAGAGGAAGGTGGTAAAGAGTGGGG | 20-70 | | 30-50 | |
| 34 | S10 | GGGGTGGGTTAAATTTTTGGGGGGGTTGTTATTTTGGGGA | 20-70 | | | |
| 35 | SB1 | TTTGGGAGGGTTGAGGTGGGGGAGGAGGAGGTAGTTAGAG | 20-70 | | 30-50 | |
| 36 | SB3 | TTTGGGCGGGTTGAGGTGGGGGAGGAGGAGGTAGTTAGAG | 20-70 | | 30-50 | |
| 37 | SB6 | TTTGGGAGGGTTGAGGCGGGGGAGGAGGAGGTAGTTAGAG | 20-70 | 41 | 30-50 | |
| 38 | SB7 | CATGGCGGGGGGGGGGGGAGAAGGGGGGGGGGGGGGTTTT | 20-70 | 43 | 30-50 | |
| 39 | SB8 | TACGGGTGGAGGGGGGGGCGGTTGGTTGTAGTTATTTGGT | 20-70 | | 30-50 | |
| 40 | SB11 | TTTGGGGGGGTTGAGGTGGGGGAGGAGGAGGTAGTTAGAG | 20-70 | 10 | 30-50 | |
| 41 | SB16 | GCTAGGGCGGGTGGAGGGTAGTTTGCGCGGGGGCGGAGGT | 20-70 | 40 | 30-50 | |
| 42 | RR58 | TTTGGGAGGGTTGAGGTGGGGGAGGCGGAGGTAGTTAGAG | 20-70 | 30 | 30-50 | |
| 43 | RR62 | TACGGGTGGAGGGGGGGGCGGTTGGTTGCAGTTATTTGGT | 20-70 | | 30-50 | |
| 44 | RR67 | TTTGGGAGGGTTGAGGTGGGGGAGGGGGAGGTAGTTAGAG | 20-70 | | 30-50 | |
| 45 | RR68 | TTTGGCAGGGTTGAGGTGGGGGAGGAGGAGGTAGTTAGAG | 20-70 | | 30-50 | |
| 46 | RR74 | GCGGGGGGTGGAAAGAGGATGAAGGGGGGTGGATTTTGGG | 20-70 | 43 | 30-50 | |
| 47 | RR80 | CATGGCGGGGGGGGGGGCAGAAGGGGGGGGGGGGGGTTTT | 20-70 | 48 | 30-50 | |
| 48 | RR81 | CATGGCGGGGGGGGGGGCAGAAGGGGGGGGGGGGGGTTTG | 20-70 | | | |
| 49 | RR83 | CATGGCGGGGGGGGGGGGAGAAGGGGGGGGGGGGGGTTTG | 20-70 | | 30-50 | |
| 50 | RR84 | GGTGGCGGGTGGGAAGAGGGATGAGGTGTGTAAGGGGGGG | 20-70 | 55 | 30-50 | |
| 51 | RR87 | GGTGGCGGTTCTATACTATCCGGGTGGGTTTAGGGGGGGT | 20-70 | 76 | 30-50 | |
| 52 | RR88 | GGTGGAGGTTCTATACTATCCGGGTGGGTTTAGGGGGGGT | 20-70 | 77 | | |
| 53 | RR89 | GGGGGGGTCGTAACTGTGGGTGGGGGAGGTGGAGGTTATA | 20-70 | | | |
| 54 | RR90 | GGGGTGGGTTCACTTTTTGGGGGGGTTGTTCTTTTGGGGA | 20-70 | | | |
| 55 | RR91 | TGGGAGGGTTGGAGGGGGTGGAGGTGAGGGTTTATTCAGA | 20-70 | | | |
| 56 | RR92 | TTTGGGAGGGCTGAGGTGGGGGAGGAGGAGGTAGTTAGAG | 20-70 | | 30-50 | |
| 57 | RR93 | TTTGGGAGGGTGGAGGTGGGGGAGGCGGAGGTAGTTAGAG | 20-70 | 114 | 30-50 | |
| 58 | RR95 | TTTGGGAGGGTTGAGGAGGGGGAGGAGGAGGTAGTTAGAG | 20-70 | | 30-50 | |
| 59 | RR96 | TTTGGGAGGGTCGAGGTGGGGGAGGGGGAGGTAGTTAGAG | 20-70 | | 30-50 | |
| 60 | RR97 | CTTGGCAGGGTTGAGGTGGGGGAGGAGGAGGTAGTTAGAG | 20-70 | | 30-50 | |
| 61 | RR98 | TTTGGGAGGGTTGAGGTGGGGGAGGAGGAGGCAGTTAGAG | 20-70 | | | |
| 62 | S1L | TAGGGAAGAGAAGGACAATGATGGTGGAGGTTATATACTA | 20-70 | | | Long |
| | | TCCGGGTGGGTTTAGGGGGGGTTTGACTAGTACATGACCA | | | | Aptamers |
| | | CTTGA | | | | |
| 63 | S2L | TAGGGAAGAGAAGGACAATGATGGGGTGGGTTAACTTTTT | 20-70 | 15 | 30-50 | |
| | | GGGGGGGTTGTTATTTTGGGGATTGACTAGTACATGACCA | | | | |
| | | CTTGA | | | | |
| 64 | S3L | TAGGGAAGAGAAGGACAATGATGGGGGGAAGGGTGGGACT | 20-70 | | | |
| | | TTAACCATACTTCGGTGGCATGTTGACTAGTACATGACCA | | | | |
| | | CTTGA | | | | |
| 65 | SB1L | TAGGGAAGAGAAGGACAATGATTTTGGGAGGGTTGAGGTG | 20-70 | 40 | 30-50 | |
| | | GGGGAGGAGGAGGTAGTTAGAGTTGACTAGTACATGACCA | | | | |
| | | CTTGA | | | | |
| 66 | SB3L | TAGGGAAGAGAAGGACAATGATTTTGGGCGGGTTGAGGTG | 20-70 | | 30-50 | |
| | | GGGGAGGAGGAGGTAGTTAGAGTTGACTAGTACATGACCA | | | | |
| | | CTTGA | | | | |
| 67 | SB5L | TAGGGAAGAGAAGGACAATGATGCTCGGGCGGGTGGAGGG | 20-70 | 37 | 30-50 | |
| | | TAGTTTGCGCGGGGGTGGAGGTTTGACTAGTACATGACCA | | | | |
| | | CTTGA | | | | |

Aptamers included have a 5′ end and 3′ end primers sequences used for amplification during SELEX. We refer to those aptamers as the Long version and we know that they bind. The sequence for the aptamers including the 5′ and 3′ primer ends is TAGGGAAGAGAAGGACAATGAT(SEQ ID NO: 159) N40TTGACTAGTACATGACCACTTGA (SEQ ID NO: 160); where N40 actual sequence of the aptamers.

TABLE 2
Other Aptamers

| | | | KD as | | Inhibition of |
|---|---|---|---|---|---|
| | | | Determined | | binding of |
| | | | by | KD as | Purified S |
| | | | ELISA | De- | protein to ACE- |
| SEQ | | | Based | termined | 2 Expressed |
| ID | | | Binding | by SPRI | on Vero |
| NO: | Name | Sequence | Assay (nM) | (nM) | E6 cells (%) |
| 68 | 1A | CCACCACACACACACACAACCACCCTACAGTCCTGTACGA | | | |
| 69 | 1B | ACACCACCACACTCATCCACCACACACACCCGCGCCTCCA | | | |
| 70 | 1C | CCACCACACACACACACAACCACCCTACAGTCCTATACGA | | | |
| 71 | 1E | CCACCACACACACACAACCACCCTACAGTCCTGTACGA | | | |
| 72 | 1F | ACCACGCCACACCCGGGACACACACGCCACAGACCTCTGC | | | |
| 73 | 1H | ACCGCACAACAACACCAAACCCAACGCTTTTGAACCGCTA | | | |
| 74 | 1J | CCCCACACACCCCCACAACACACCACTCACGGCTCCCCA | | | |
| 75 | 2B | GCACACCACCACCGCGAGAGCCCCCGACCTGCACCCCTGC | | | |
| 76 | 2C | CACACACACCCCTGGCTCCAAACCACCCAACCTTTGGCGA | | | |
| 77 | 2D | CAGGCAACAACACACCACGCGCACACACCTTTACGTCACA | | | |
| 78 | 2E | CACCACCACACCTACGCTCCAATTACCTACCCCGGGCCCC | | | |
| 79 | 2F | CACACACACCCCTGGCTCCAAACCACCCAACCTTCGGCGA | | | |
| 80 | 2H | ACCACACACCACGAGATTTAGGGCGCCACACCACTCCGTC | | | |
| 81 | 21 | CAACAAGCACACACTCACTACACACTCACACACGCCTTGG | | | |
| 82 | 3B | CACCGCAGGATACTACACACCAGCACACAGCCACCACACA | | | |
| 83 | 3D | ACCACCACGCACCACACCACCACACCGAGACTGCCCCACG | | | |
| 84 | 3E | ACACACACCCACCTAGCGCAGGACACACCAGTATCCACCC | | | |
| 85 | 3G | GCACACCACACAACCAAAAGCAAGGAACCAATACCCACCG | | | |
| 86 | 3H | CCAACACCATACACAGCACACAACCAGAGACGCCACATGA | | | |
| 87 | 3 | AGCAGCCCAACACATCCACACTCCCACAGCCCCTCTCCTG | | | |
| 88 | 3J | GAGCAGCCAACAACAAAACAAAGAACAGCACACCACAGCC | | | |
| 89 | 4A | CACACGCACACACGACACAAAACCGGATAACACAGCACTG | | | |
| 90 | 4B | GCCAGGCCACAGCACTACACACCACCACACACCGGCCAGA | | | |
| 91 | 4C | CCCACACCCACGCGCGGAACCACCCACACACTCACCCAGC | | | |
| 92 | 4F | CACCACACGCACCACCCTCCACCCCGCCCCAGAGCCGCCG | | | |
| 93 | 4G | CACACACACACCAACAGCCACTGCTAACTCTCCTAGG | | | |
| 94 | 4I | CACCACACACATCACCCACACGACGCCAACACAGCGCGGT | | | |
| 95 | 1 | CCACCACACACACACACAACCACCCTACAGTCCTGTACGA | | | |
| 96 | 2 | ACACCACCACACTCATCCACCACACACACCCGCGCCTCCA | | | |
| 97 | 3 | CCACCACACACACACACAACCACCCTACAGTCCTATACGA | | | |
| 98 | 4 | CCACCACACACACACAACCACCCTACAGTCCTGTACGA | | | |
| 99 | 5 | ACCGCACAACAACACCAAACCCAACGCTTTTGAACCGCTA | | | |
| 100 | 6 | ACACCCCCACCACCCTTGCACACATCACACTCCACACGCA | | | |
| 101 | 9 | CCAACACCATACACAGCACACAACCAGAGACGCCACATGA | | | |
| 102 | 10 | AGCAGCCCAACACATCCACACTCCCACAGCCCCTCTCCTG | | | |
| 103 | 12 | CACCACACGCACCACCCTCCACCCCGCCCCAGAGCCGCCG | | | |
| 104 | 13 | CACACACACACCAACAGCCACTGCTAACTCTCCTAGG | | | |
| 105 | 14 | GCCCAACCGATCAACACACACATACACACACATTGGATCA | | | |
| 106 | 15 | CACACACACACACGAACAACCAACAATCTCGTAGCGGCTA | | | |
| 107 | 16 | CACACACACACACGCACATCATCATGGTTAGTCTGATTCA | | | |
| 108 | 17 | CGGGGCAGAACACACACACTACAGGACACACACTTCGTTAC | | | |
| 109 | 18 | CACACACACACACACAACAAAGAGTGCTAAAGACGGCAGC | | | |
| 110 | 19 | CACACACACGGACACACACGGTACAATATCACGGAGAGTC | | | |
| 111 | 20 | GCAGCAAGGAGTAGACTCAACACACACACACACTACCCTG | | | |
| 112 | 21 | CACACACACACACGCTTGGATTTAACACGCCAGGACAATG | | | |
| 113 | 22 | CACACACATACACACACACAACTTGCTTATTCGGCGAAGT | | | |
| 114 | 23 | CACACACACGCACACTGACATTCAAGGCTATTACGCTCTA | | | |
| 115 | 26 | CACACACACACACACACAGTCGGGTAAACTCCTCCATTTG | | | |
| 116 | 27 | CACACACACACACGAACCATATAACATTGCAGTTGCCTAG | | | |
| 117 | 29 | CAGGCAACAACACACCACGCGCACACACCTTTACGTCACA | | | |
| 118 | 30 | CACACACACACACACTGACAGTACATCGGTTAGTATCCGG | | | |
| 119 | 31 | GACCACACACCCAAACCACACACACTTAACACACACCGTA | | | |
| 120 | 33 | CACACACACACATGCACACCCTTACTTAGCTTGGCGACGT | | | |
| 121 | 34 | GGACCACGATACACACACACACTATACACTTGGGCGATGA | | | |
| 122 | 35 | CACACAACACACACCACACAGTTAAAGATGATTCGGGGCT | | | |
| 123 | S3 | GGGGGGAAGGGTGGGACTTTAACCATACTTCGGTGGCATG | | | |
| 124 | S6 | CAAGTGCATGTCGTGCGTCGGGTAGATTGGGTGGGTTGGG | | | |
| 125 | S7 | GGGGGGGGTAAAGAGAGGCGTTAAAGTGGCGTTGTTGGTC | | | |
| 126 | SB2 | TTGAATAGCGCCAGTCGTAATCATCGATATGGGACTGTAA | | | |
| 127 | SB4 | CACGTGCATGTCGTGCGTCGGGTAGATTGGGTGGGTTGGG | | | |
| 128 | SB5 | GCTCGGGCGGGTGGAGGGTAGTTTGCGCGGGGGTGGAGGT | | | |
| 129 | SB9 | CAAGTGCATGTCGTGCGTCGGGTAGATTGGGCGGGTTGGG | | | |
| 130 | SB10 | GTGGGTGGGATATTGGTGGTGGTGCGCTAAAGTGTATTGG | | | |
| 131 | SB17 | GGGGGGGGTAGATGCGGTAGGCCGGTAGGCATTCAATGGT | | | |
| 132 | RR51 | TTGACTAGCGCCAGTCGTAATCATCGATATGGGACTGTAA | | | |
| 133 | RR52 | TTGAATAGCGCCAGTCGTAATCATCGATATGGGACTGTAC | | | |
| 134 | RR53 | TTGAATAGAGCCAGTCGTAATCATCGATATGGGACTGTAA | | | |
| 135 | RR54 | TTGAATAGCGACAGTCGTAATCATCGATATGGGAATGTAA | | | |
| 136 | RR55 | TTGAATAGCGACAGTCGTAATCATCGATATGGGACTGTAA | | | |
| 137 | RR56 | TTGAACAGCGCCAGTCGTAATCATCGATATGGGACTGTAA | | | |
| 138 | RR57 | TTGAATAGCGCCAGTCGTAATCATCGATATGGGAATGTAA | | | |
| 139 | RR59 | TTTAATAGCGCCAGTCGTAATCATCGATATGGGACTGTAA | | | |
| 140 | RR60 | TTGAATAGCGCCAGTCGCAATCATCGATATGGGACTGTAA | | | |
| 141 | RR61 | TTGAGTAGCGCCAGTCGTAATCATCGATATGGGACTGTAA | | | |
| 142 | RR63 | CTGAATAGCGCCAGTCGTAATCATCGATATGGGACTGTAA | | | |
| 143 | RR64 | TTGACTAGCGACAGTCGTAATCATCGATATGGGACTGTAA | | | |
| 144 | RR65 | TTGAATAGCGCCAGTCGTAATCATCGATATGGGAGTGTAA | | | |
| 145 | RR66 | TTGACTAGAGCCAGTCGTAATCATCGATATGGGACTGTAA | | | |
| 146 | RR69 | TTGAATAGGGCCAGTCGTAATCATCGATATGGGACTGTAA | | | |
| 147 | RR71 | TTGAATAGTGCCAGTCGTAATCATCGATATGGGACTGTAA | | | |
| 148 | RR72 | TTGAATAGCGTCAGTCGTAATCATCGATATGGGACTGTAA | | | |
| 149 | RR73 | TTGAATAGCGCCAGTCGTAATCATCGATATAGGACTGTAA | | | |
| 150 | RR75 | GCTAGGCTTACGTAGGGAACTCGTACCGTGGTTAGCAATC | | | |
| 151 | RR76 | TTGAATAGCGCCAGTCGTAATCATCGCTATGGGACTGTAA | | | |
| 152 | RR77 | TTGAATAGCGCCAGTCGTAATCATCGATATGCGACTGTAA | | | |
| 153 | RR79 | CTCGACTCGTACGCCAACTTAAACTGAGTTGTTGCGCCCC | | | |
| 154 | RR82 | CATGGCGGGTGGGGGGGCAGAAGGGGGGGGGGGGGGTTTT | | | |
| 155 | RR85 | GTGGGGGGGGTTGCAAAGGTTAGAGGTGTAGGGAGGTGGT | | | |
| 156 | RR94 | TTTGGGAGGGTAGAGGTGGGGGAGGCGGAGGTAGTTAGAG | | | |
| 157 | RR99 | TTTGGGAGGGTGGAGGTGGGGGAGGAGGAGGTAGTTAGAG | | | |
| 158 | RR100 | TTTGGGAGGGTTGAGGGGGGGGAGGAGGAGGTAGTTAGAG | | | |
| 165 | AYA2012002_ | TAGGGAAGAGAAGGACAATGATTTTGGGAGGGTTGAGGCGG | | | |
| | L | GGGAGGAGGAGGTAGTTAGAGTTGACTAGTACATGACCAC | | | |
| | | TTGA | | | |
| 166 | AYA2012003_ | TAGGGAAGAGAAGGACAATGATTACGGGTGGAGGGGGGGGC | | | |
| | L | GGTTGGTTGTAGTTATTTGGTTTGACTAGTACATGACCAC | | | |
| | | TTGA | | | |
| 167 | AYA2012006_ | TAGGGAAGAGAAGGACAATGATTTTGGGGGGGTTGAGGTGG | | | |
| | L | GGGAGGAGGAGGTAGTTAGAGTTGACTAGTACATGACCAC | | | |
| | | TTGA | | | |
| 168 | AYA2012007_ | TAGGGAAGAGAAGGACAATGATCAAGTGCATGTCGTGCGTC | | | |
| | L | GGGTAGATTGGGCGGGTTGGGTTGACTAGTACATGACCAC | | | |
| | | TTGA | | | |
| 169 | AYA2012008_ | TAGGGAAGAGAAGGACAATGATCATGGCGGGGGGGGGGGGA | | | |
| | L | GAAGGGGGGGGGGGGGGTTTTTTGACTAGTACATGACCAC | | | |
| | | TTGA | | | |
| 170 | AYA2012009_ | TAGGGAAGAGAAGGACAATGATGCTCGGGCGGGTGGAGGGT | | | |
| | L | AGTTTGCGCGGGGGTGGAGGTTTGACTAGTACATGACCAC | | | |
| | | TTGA | | | |

Aptamers 165-170 included have a 5′ end and 3′ end primers sequences used for amplification during SELEX. We refer to those aptamers as the Long version and we know that they bind. The sequence for the aptamers including the 5′ and 3′ primer ends is TAGGGAAGAGAAGGACAATGAT(SEQ ID NO: 159) N40TTGACTAGTACATGACCACTTGA (SEQ ID NO: 160); where N40 actual sequence of the aptamers.

### Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages). Peptides Variants

As discussed herein there are numerous variants of the aptamers (i.e. SEQ ID Nos: 16-30) disclosed herein that are known and herein contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 5 and 6 and are referred to as conservative substitutions.

TABLE 5
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 6
Amino Acid Substitutions Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 5, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 5 and Table 6. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH-(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂-); Holladay et al. Tetrahedron. Lett 24:4401-4404 (1983) (—C(OH)CH₂-); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. Nucleic Acids

There are a variety of molecules disclosed herein that are nucleic acid based, including for example the nucleic acids set forth in SEQ ID Nos: 1-158 and/or 162-170, or any fragments thereof. The disclosed nucleic acids are made up of for example, nucleotides, nucleotide analogs, or nucleotide substitutes. Non-limiting examples of these and other molecules are discussed herein. It is understood that for example, when a nucleic acid is DNA, the DNA will typically be made up of Adenine (A), Cytosine (C), Thymine (T), and Guanine (G). Similarly, when a nucleic acid is RNA, the RNA will typically be made up of A, C, G, and uracil (U). Likewise, it is understood that if, for example, an antisense molecule is introduced can be advantageous that the antisense molecule be made up of nucleotide analogs that reduce the degradation of the antisense molecule in the cellular environment.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (.psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to-O[(CH₂)ₙ O]ₘ CH₃, —O(CH₂)ₙ OCH₃, —O(CH₂)ₙ NH₂, —O(CH₂)˜CH₃, —O(CH₂)˜-ONH₂, and —O(CH₂)ₙON[(CH₂)ₙ CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

It is also understood in a nucleotide substitute that both the sugar and the phosphate moieties of the nucleotide can be replaced, by for example an amide type linkage (aminoethylglycine) (PNA).

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

As noted herein, the isolated nucleic acids can be modified to comprise an substitution, insertion, or deletion of one or more nucleotides in the disclosed nucleic acid aptamer sequences set forth in Table 1, 2, and 3, such as, for example, SEQ ID NO: 1-158 and/or 162-170, or any fragment, derivative, or variant thereof comprising at least 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto. In one aspect, the truncation can comprise a deletion of 1, 2, 3, 4, 5, 6, 7, 8, or 9 nucleotides from the 3′ or 5′ end of the aptamer. As disclosed herein, the 5′ end is not as essential as the 3′-end of the nucleotide. Thus, disclosed herein are isolated nucleic acids wherein the nucleic acid sequence is a truncation at the 5′ end. In one aspect, disclosed herein are isolated nucleic acids wherein the nucleic acid sequence is a truncation of SEQ ID NO: 1-158 and/or 162-170.

The S protein-specific aptamers disclosed herein (such as for example, any of the nucleic acids encoding an amino acid as set forth in SEQ ID NOs: 1-158 and/or 162-170, or any fragment, derivative, or variant thereof comprising at least 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto, including any of the nucleic acids set forth in Table 1, 2, and 3 can be used in diagnostics, therapeutic and theranostic purposes. The disclosed aptamers have advantages over prior technologies including: (1) integrity—more stable biological probe than antibodies—in terms of biochemical resistance to change in the normal range of temperature, pressure, and chemical/biochemical exposure; (2) lower to absent immunogenicity (especially important for therapeutic purposes); (3) much simpler to synthesize—generally requires fewer steps resulting to better synthetic efficiency and purity, (4) less expensive to produce; (5) longer shelf-life—amenable to longer-term storage because of inherent stability; (6) faster results (within a few minutes); and (7) some configurations do not require an instrument and, therefore, are amenable to point-of-care clinical applications.

In one aspect, disclosed herein are methods of detecting a viral infection in a subject comprising obtaining a biologic sample from the subject and measuring the concentration of a viral protein (such as, for example a coronavirus S protein) in the subject using one or more of the nucleic acids, compositions, or kits disclosed herein (such as for example, any one or combination of two or more of the nucleic acids disclosed in SEQ ID NOs: 1-158 and/or 162-170 of Tables 1, Table 2, and Table 3 or combinations disclosed in Table 4 or compositions comprising said nucleic acids). For example, disclosed herein are methods of detecting a viral infection in a subject comprising obtaining a biologic sample from the subject and measuring the concentration of a viral protein in the subject using one or more of the nucleic acids, compositions, or kits of any preceding aspect, wherein the viral infection is selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, A246-252, L452Q, F490S, D614G, and T859N mutations in C.37 (Lambda), SARS-CoV-2 with T478K, Q498R, and H655Y mutations in B.1.1.529 (Omicron)), or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Due to the application in detection of S protein and/or detection of a clinical indication implicated by the presence of S protein (such as, for example, coronaviral infection), it is understood and herein contemplated that modification of the disclosed aptamers (including any nucleic acid encoding the peptides set for in SEQ ID Nos: 1-158 and/or 162-170 to comprise a detectable tag such as, for example, a latex bead, magnetic bead, fluorescence labels; fluorescent probes, chemiluminescent labels, radiolabels, and/or nanoparticle probe.

As used herein, a label or tag can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein-(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy F1; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson −; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; ; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the aspect include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

Additionally, the interaction of the aptamer with protein (i.e, S protein) can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

The aptamers disclosed herein and their derivatives are demonstrated to have preferential binding to coronavirus S protein in solution, whole blood, blood sera, and in blood plasma in the relevant physiological concentration range. Quantitative, semi-quantitative, and qualitative methods that may or may not require separate equipment have been shown to give test values in concordance with current protocols approved for clinical use. This makes the aptamers capable of being used to quantify the level of S protein in the blood and other appropriate samples. The disclosed nucleic acids can also be used to probe S protein in biological samples and in vivo settings—a property that can be extended to diagnostic and therapeutic applications. Quantitative, semi-quantitative, and qualitative methods that do or do not require separate equipment have been shown to give test values in concordance with current protocols approved for clinical use.

Therapeutic applications involving the same DNA/RNA sequences are amenable for in vivo testing and could be formulated for therapeutics based on its targeting nature and unique sequence. The isolated nucleic acids aptamers disclosed herein (such as any (including any nucleic acid encoding the peptides set for in SEQ ID Nos: 1-158 and/or 162-170, as well as, any fragment, derivative, or variant thereof comprising at least 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto.) can be used to deliver chemical species or physiologically relevant payloads. In one example, the aptamer is expected to hone in on areas where clotting is dominant and deliver anticoagulant species. Other coagulation factors could be delivered to the site where it is necessary, thereby lowering the required dosage because of site-specific action. Accordingly, disclosed herein are methods of treating, inhibiting, reducing, decreasing, ameliorating, and/or preventing a viral infection in a subject comprising administering to the subject one or more of the nucleic acids or compositions disclosed herein (such as for example, any one or combination of two or more of the nucleic acids disclosed in SEQ ID NOs: 1-158 and/or 162-170 of Tables 1, 2 and 3 or combinations disclosed in Table 4 or compositions comprising said nucleic acids), as well as, any fragment, derivative, or variant thereof comprising at least 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto.).

TABLE 4
Aptamer Mix

| | | Inhibition of binding |
|---|---|---|
| | | of Purified S protein |
| | | to ACE-2 Expressed |
| | Mix | on Vero E6 cells (%) |
| Mix1 | S10, S11, SB1L, SB3L, SB5L, RR68, | 30-50 |
| | RR74, RR80 | |
| Mix2 | S9, S11, SB7, SB8, SB11, S1, SB16 | 30-50 |
| Mix3 | S10, RR74, S2L | 30-50 |
| Mix4 | S1L, S2L, S11, RR80, RR83 | 30-50 |
| Mix5 | S4, S5, S12, SB6, RR84, RR87, | 30-50 |
| | RR92, RR93 | |

Accordingly, the treatment can involve the administration of a combination of two or more nucleic acids from SEQ ID NOs: 1-158 and/or 162-170 either separately or in combination in a single composition (for example, any of the combinations disclosed in Table 4). Thus, in one aspect, disclosed herein are methods of treating, inhibiting, reducing, decreasing, ameliorating, and/or preventing a viral infection in a subject, wherein the one or more nucleic acids are selected from the group consisting of a) S10, S11, SB1L, SB3L, SB5L,RR68, RR74,RR80; b) S9, S11, SB7, SB8, SB11, S1, SB16; c) S10, RR74, S2L; d) SIL, S2L, S11, RR80, RR83; and/or e) S4, S5, S12, SB6, RR84, RR87, RR92, RR93.

The disclosed methods can be used to treat any viral infection. For example, disclosed herein are methods of treating, inhibiting, reducing, decreasing, ameliorating, and/or preventing a viral infection in a subject comprising administering to the subject one or more of the nucleic acids or compositions disclosed herein (such as for example, any one or combination of two or more of the nucleic acids disclosed in SEQ ID NOs: 1-158 and/or 162-170 of Tables 1, 2, and 3 or combinations disclosed in Table 4 or compositions comprising said nucleic acids), wherein the viral infection is selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, A246-252, L452Q, F490S, D614G, and T859N mutations in C.37 (Lambda), SARS-CoV-2 with T478K, Q498R, and H655Y mutations in B.1.1.529 (Omicron)), or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2. In some aspects, the nucleic acid binds to the S protein of a coronavirus (such as, for example, binding to the S protein that inhibits binding of the S protein to the ACE2 receptor). Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for detecting the presence of a viral infection (such as, for example, a coronaviral infection) or simply an S protein of a coronavirus comprising any nucleotide encoding the amino acids set forth in SEQ ID Nos: 1-158 and/or 162-170, as well as any fragment, derivative, or variant thereof comprising at least 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto.

While various embodiments of the present disclosure have been described above, it should be understood that they have been presented by way of example only, and not of limitation. Likewise, the various diagrams may depict an example architectural or other configuration for the invention, which is provided to aid in understanding the features and functionality that can be included in the invention. The invention is not restricted to the illustrated example architectures or configurations, but the desired features can be implemented using a variety of alternative architectures and configurations.

Indeed, it will be apparent to one of skill in the art how alternative functional configurations can be implemented to implement the desired features of the present disclosure. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present invention should not be limited by any of the above-described exemplary embodiments.

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. Example 1

### (1) Aptamers

Aptamers are RNA or DNA oligonucleotides that, through their 3-dimensional structures, bind to specific target molecules with high affinity and specificity similar to antibodies. Aptamers have a lot of advantages over antibodies: they are synthetically created reducing the cost and time of production, there is no lot-to-lot variability, they are stable at room temperature, they are smaller than antibody proteins and can easily be modified chemically.

The target molecules of aptamers can be small molecules, large biomolecules, and even cells. Since their advent in 1990, aptamers have been developed for use in diagnostics. Aptamers specific to the Coronavirus S protein are disclosed herein. The development of the testing platform and validation of the diagnostic protocols or device which has always been the bottleneck for clinical diagnostics is also disclosed herein. The inherent high affinity, specificity, biochemical stability, low to absent immunogenicity, fast production, and relatively low cost of production makes aptamers very desirable as substitutes for antibodies in diagnostics and future therapeutic applications. Aptamers are often referred to as “synthetic” antibodies.

### (2) SELEX

A combinatorial process called SELEX (systematic evolution of ligands by exponential enrichment) has been developed in the Gold Laboratory (Univ. of Colorado, Boulder) in 1990. It is a systematic selection process that combines the power of biochemical selection with polymerase-chain reaction (PCR) through a series of binding-elution processes and nucleic acid amplifications. It amplifies the DNA or RNA after an elution cycle involving binding to immobilized targets. The repeated amplification of only the eluted target-binding sequences—initially from a pool of 10¹² to 10¹⁶ different nucleic acids after an elution step—allows for the selective amplification of the strongest binding nucleic acids.

On December 2004, the U.S. Food and Drug Administration (FDA) awarded OSI Pharmaceuticals the first aptamer-based drug for the treatment for age-related macular degeneration (AMD), called Macugen™. In addition, the company NeoVentures Biotechnology Inc. has successfully commercialized the first aptamer based diagnostic platform for analysis of mycotoxins in grain. Many contract companies develop aptamers and aptabodies to replace antibodies in research, diagnostic platforms, drug discovery, and therapeutics such as Aptagen LLC and Base Pair Biotechnologies.

### b) General Two-Step Coupling Procedure:

Covalent Coupling of Oligonucleotides to Carboxylate-Modified Particles Via—COOH+-NH2 Binding. The first step is run at an acid pH to ensure that carboxylic acid groups are in COOH form. The second step is run at basic pH to ensure that amine groups are in NH2 form.

### (1) Reagent Preparation

Nanosphere Beads comprising Carboxylate-modified polystyrene 4% solids are prepared in water. Water-Soluble Carbodiimide (WSC) Solution comprising no more than 2% (w/v) 1-ethyl-3-(3dimethylaminopropyl) carbodiimide (Sigma Chem. Co.) solution freshly prepared in deionized water. Other reagents include a pre-activation buffer comprising 0.05M KH2PO4, pH 4.5, a coupling buffer comprising 0.2M Borate Buffer, pH 8.5; an Oligonucleotide Solution: Calculated*volume of 100 uM nucleotide solution in Coupling (Buffer to calculated*final volume); a quenching Solution comprising 5 mM ethanolamine; and a Wash/Storage/Dialysis Buffer comprising PBS (pH 7.4) or 0.2 M Borate Buffer, pH 8.5 with 0.05% NaN3)

### (2) Washing/Pre-Activation

To 1 ml microsphere suspension, add Pre-Activation Buffer. Place suspension on a magnet stirrer and maintain at room temperature (˜23° C.). Add WSC Solution (as calculated; may be diluted depending on starting amount of the nanosphere latex beads). NOTE: Add slowly at first attempt and stop as soon as coagulation or sudden cloudiness occurs. Note the volumes used (for next batch) Allow to react for 2 to 3 hours.

### (3) Washing/Protein Coupling

Wash particle suspension in saline, and resuspend in 5 ml saline. Add equal volume of microsphere suspension to calculated equivalent*volume of Oligonucleotide Solution. Incubate at 22° C. for 20 hours (or at least overnight).

### (4) Blocking/Washing

To neutralize surface carboxyl groups that are not bound to avidin, add Quenching Solution (Ethanolamine, 5 nM); may then add BSA (blocker) to a suitable concentration (no more than 2% w/v). Dialyse Latex-Aptamer suspension once overnight in Wash/Storage/dialysis Buffer at room temperature. Centrifuge at 15,000×g if needed (to remove coagulated spheres) or filter using 0.2 to 1 um filter, retaining the supernate. Example 2: S Protein Aptamer Selection Protocol

The following protocol was used in the SELEXbased screening for the S protein Aptamers: S protein

### a) 1. Immobilization of the Target Protein on Br-CN Activated Sepharose.

S protein was immobilized on Br-CN activated sepharose at final concentration 1.5 mg of protein per 1 ml sepharose. Binding of S protein was quantitated and confirmed using Pirce BCA protein Assay kit (cat #23227)

### b) 2. Aptamers Selection

1. Dilute 10 nmole Aptamer library (1 tube TriLink biotechnologies) in 100 ul of DNase and RNase free water. Save 1 ul of the library for PCR (original library)
2. Add 100 ul of binding buffer (50 mM Tris pH 7.5, 150 mM NaCl, and 1 mM EDTA) to the diluted library and heat it at Hybex at 950 C for 10 min.
3. Cool down the library on ice for 10 min
4. Dilute the ssDNA library in 1 ml total of binding buffer. Save 5 ul for PCR (loading library)
5. Wash 100 ul of the sepharose beads (bed volume) alone, or the S protein beads with 1 ml of the binding buffer 3 times (5 min. rotation between each wash)
6. Apply diluted library to the sepharose beads (negative selection), incubate for 30 min. at room temperature and collect the flow throw. Save 5 ul for PCR.
7. Apply the flow throw after negative selection to S protein sepharose beads and incubate for 2 hours at room temperature.
8. Collect flow throw, save 5 ul for PCR.
9. Wash the beads 3 times with the binding buffer and collect the washes for PCR.
10. Elute the DNA library from the S protein beads:
   i. Add 30 μmole of NaOH (0.2 ml of 0.15M stock), gently vortex and rotate at RT for 10 mins to elute the ssDNA from S protein
   ii. The tube is spun down and the unretained ssDNA is added into a tube containing 30 μmole of acetic acid (0.2 ml of 0.15M stock) to neutralize the base.
   iii. Buffer the solution by adding 40 μl of 3 M sodium acetate followed by 1 ml of cold 100% ETOH to precipitate the ssDNA.
   iv. Repeat the NaOH elution process on the beads and precipitate the eluted ssDNA
   v. Place the two tubes in −20c for 2 h or overnight
   vi. Spin at 13,200 rpm for 45 mins at 40 C
   vii. Wash with 1 ml of 70% ETOH and
   viii. Spin for 20 mins at 13,200 rpm at 40 C (repeat the wash 2×)
   ix. Remove ETOH and air dry (10 min) or speed vacuum dry
   x. Reconstitute each tube in 15 μl of water, incubate at 370 C on Hybex for 10 min, vortex and spin down.

### c) PCR Amplification

PureTaq ready to go PCR Beads are lyophilized and need to be hydrated in a total 25 volume of 25 μl. Add 5 μl of each of the forward and reverse primers supplied by Trilink (10 μM stock), 1 μl or 5 ul of the saved samples and H₂O up to 25 μl. Set up the following PCR program:
1. 95c for 5 mins
2. 95 c for 30 sec
3. 50 c for 30 sec
4. 72c for 30 sec repeat steps 2-4 25 times
6. 72 c for 5 mins hold at 4 c

Analyze PCR product using Bioanalazer. Average size of the aptamers is 80 nucleotides. PCR product of elution was collected. The combined PCR product is used for the second round of selection. Example 3: Small-Fragment DNA/RNA—Aptamer Structures and Separation

Secondary Structure Variations in Aptamer Folding just like any polymer, the folding(s) of DNA and RNA are dictated by their sequence and environmental conditions. The permutations of bond-rotations and interactions increase exponentially as the oligonucleotide sequence length increases. The simulation of the candidate aptamers' secondary structure folding is, therefore, mandatory to help explain and (simulate) tertiary structures and concomitant binding—specificities, affinities and all.

Examination of Folding Isomers (“Foldamer”) Rationale: The fact that DNA and RNA fragments fold to minimize energy into different possible configurations indicate that such aptamers be analyzed in that regard. The more different the aptamer candidates' foldamers are in terms of secondary (and therefore, 3D) structure and the greater their differences in corresponding energies, the more varied their binding affinities are expected. As such, the possibility of the isolation and study of the active, if not the most active, foldamer(s) must be ascertained (if at all necessary). Structures that have very close energy levels can easily interchange in solution, therefore, the foldamers can be, in practical considerations, equivalent as they can interconvert without energy input or assistance.

Only a few techniques are known to be able to resolve DNA sequences with one base pair difference (deletion or substitution). Even more elusive to find and validate is a technique that can resolve a specific secondary structure of the same DNA sequence. Varying attempts have been made to solve such a problem. Besides Nuclear magnetic Resonance, only two techniques are published to have been used successfully to study phenomena in solution and only two that involve and/or close to achieving foldamer separations: electrophoresis and HPLC. Example 4: High-Affinity Neutralizing DNA Aptamers Against SARS-CoV-2 Spike Protein Variants

Coronavirus disease 2019 (COVID-19) is a disease caused by severe acute respiratory syndrome coronavirus 2 (SARS-CoV-2). Ever since it emerged in December 2019, SARS-CoV-2 has caused millions of infection cases and deaths and the greatest global public health and economic crisis around the world and the United States.

SARS-CoV-2 is a single-stranded RNA-enveloped virus whose genome encodes four major structural proteins, including the spike (S), membrane (M), envelope (E), and nucleocapsid (N) proteins. Among them, the S protein plays a key role in the receptor recognition (S1 unit), and cell membrane fusion processes (S2 unit), and determines the infectivity of the virus and its transmissibility in the host. The S1 contains the receptor binding domain (RBD), which is mainly responsible for binding the virus to the angiotensin-converting enzyme 2 (ACE2) receptor. Once the RBD binds to the host ACE2 receptors, the cell surface serine protease TMPRSS2 starts to promote SARS-CoV-2 viral uptake and fusion at the cellular level. The virus enters the host cell and releases the viral RNA into cytoplasm, after which it expresses and replicates its genomic RNA in order to make full-length copies that are integrated into freshly produced viral particles. In addition, the S protein contains a site that is recognized and is activated by furin, a host cell enzyme expressed in various human organs, such as the liver, the lungs, and the small intestines. Because of S protein's specific structure, it allows the SARSCoV-2 viruses to bind at least 10 times more tightly to ACE2 receptors than the corresponding S protein of other SARS viruses and can potentially attack several organs at the same time.

The biggest challenge to fighting COVID-19 is the lack of specific anti-SARS-CoV-2 drugs that target different variants. The common strategy of current COVID-19 treatment is “old drug, new use”. Among these drugs are RNA dependent RNA polymerase inhibitors (e.g. Remdesivir, Favipiravir, Ribavirin and interferons), protease inhibitors (e.g. Lopinavir and Ritonavir), hydroxychloroquine, azithromycin, monoclonal antibody, or convalescent plasma. Most of these potential drugs are being investigated for their safety and efficacy against COVID-19. Remdesivir has shown to be the most promising and hopeful anti-viral therapeutic, while many other drugs have shown severe side effects, uncertain benefits, or contraindicated conditions Recently, the U.S. FDA has authorized Pfizer's Paxlovid for emergency use within five days of symptom onset, but not for pre-exposure or postexposure prevention of COVID-19. At the same time, the FDA noted that Paxlovid may result in significant drug interactions. On the other hand, many research laboratories are continuing to study and develop new drugs against COVID-19. Neutralizing antibodies for the prevention and treatment of COVID-19 is one of the fields that focuses on the N-terminal domain (NTD) and receptor-binding domain (RBD) of the S protein. This includes humanized monoclonal antibodies, antibodies cloned from human B cells, and single-chain camelid antibodies. However, coronaviruses are RNA viruses that continue to mutate, evolve, and hence develop resistance to drugs easily. So far, different mutations have been found in all four structural proteins and other viral proteins that may lead to escape from antibody recognition, resulting in antibodies that have a weaker effect or no effect at all for the new variant types of coronavirus. For example, SARS-CoV-2 variants that include mutations of A475V, L452R, V483A, F490L, and N234Q can become resistant or markedly resistant to some neutralizing antibodies.

More than that, antibody-based vaccines and therapeutics could potentially increase the risk of exacerbation of COVID-19 severity through antibody dependent enhancement (ADE), which may lead to an increase of unwanted immune reactions, virus infectivity, and virulence. Although the relevance of in vitro ADE for human coronaviruses remains less clear, several viruses, including human immunodeficiency virus (HIV), Ebola, and influenza have been well documented. Wan et al. showed that neutralizing monoclonal antibody against the RBD of MERS-COV increased the uptake of virion into macrophages and various cell lines transfected with FcγRIIa (Fc gamma receptor IIa). Thus, to address these limitations, it is urgent and necessary to develop an effective anti-SARS-CoV-2 drug to inhibit viral infection with less side effects.

Aptamers are short, single-stranded RNA or DNA molecules that have a high affinity for specific target molecules. Aptamers are often referred to as chemical antibodies because their interaction with their target is similar to that of an antigen antibody interaction. However, aptamers have many advantages over antibodies, such as smaller size, lower immunogenicity, long shelf life and stability, less batch-to-batch variation, ease of modification, cost-effectiveness, and short production time. Moreover, aptamers' flexible 3-dimensional structures allow them to fold around the complex surfaces of their target molecules, facilitating greater flexibility in selecting aptamers for various targets such as peptides, proteins, small organic compounds, toxins, cells, viruses, and bacteria, etc. Recently, several groups have identified DNA aptamers that recognize the S protein of SARS-CoV-2. The majority of them were focusing on developing aptamers that bind RBD or S1 domain of S protein.

In this study, we report the development of a series of single-stranded DNA (ss-DNA) aptamers targeting the trimer S proteins of Wuhan original strain. Selected aptamers were studied for their binding affinity and inhibitory efficacy. The truncation approach was used to improve the binding capacity of aptamers as well as their inhibitory efficiency to prevent the binding of trimer S protein to the ACE2 receptors. The results show that our aptamers were not only able to bind to the trimer S protein of the Wuhan original strain, but also bind multiple variants of trimer S proteins of Delta, Delta plus, Alpha, Lambda, Mu, and Omicron and inhibit their binding to ACE2 receptors in Vero E6 cell line. To further analyze the inhibitory efficacy of the selected aptamers, we used virus-like particles (VLPs) packaged with Green Fluorescent Protein (GFP) plasmid to mimic the SARS-CoV-2 virus. Our modified aptamers AYA2012004 L, AYA2012004 L-M1, and AYA2012004 L-M2 showed up to 70% inhibition of the binding of virus-like particles (VLPs) expressing S protein to ACE2 receptor expressed in Human Embryonic Kidney 293T (HEK293T) cells that overexpress ACE2 receptors. Overall, the findings indicate that our reported aptamers are an innovative therapy for the treatment of COVID-19. They hold many advantages over existing therapies due to better efficacy, the ability to identify different variants of SARS-CoV-2 and safety.

### a) Materials and Methods

### (1) Reagents

Human ACE2ACEH protein, Fc-tag (#AC2-H5257); SARS-CoV-2 spike protein trimer, His-tag Wuhan strain (#SPN-C52H2); SARS-CoV-2 S protein, His Tag, Super stable trimer Wuhan strain (#SPN-C52H9, SPN-C52H7); SARS-CoV-2 S protein (HV69-70del, Y144del, N501Y, A570D, D614G, P681H, T716I, S982A, D1118H), His Tag Alpha strain (#SPN-C52H6); SARS-CoV-2 Spike Trimer (T19R, G142D, EF156-157del, R158G, L452R, T478K, D614G, P681R, D950N), His Tag Delta strain (#SPN-C52He); SARS-CoV-2 Spike Trimer (G75V, T76I, SYLTPGD 247-253 del, L452Q, F490S, D614G, T859N), His Tag Lambda strain (#SPNC52Hs); SARS-CoV-2 Spike Trimer (T19R, V70F, FR157-158Del, A222V, W258L, K417N, L452R, T478K, D614G, P681R, D950N), His Tag Delta plus strain (#SPNC52Ht); SARS-CoV-2 Spike Trimer (T95I, Y144S, Y145N, R346K, E484K, N501Y, D614G, P681H, D950N), His Tag Mu strain (#SPN-C52Ha) and SARS-CoV-2 Spike Trimer, His Tag (B.1.1.529/Omicron) strain (#SPN-C52 Hz); anti-SARSCoV-2 RBD neutralizing antibody, human IgGl(#SAD-S35) were purchased from ACRO Biosystems; DNA Polymerase kit for PCR (#170-8870) was from Bio-Rad; ssDNA library consisting of 40 random nucleotides that are flanked by two 23 bases of primer sequences (5′ TAGGGAAGAGAAGGACATATGAT)(SEQ ID NO: 161) (N40) TTGACTAGTACATGACCACTTGA 3′(SEQ ID NO: 160), #0-32140-10), forward, reverse and 5′biotin reverse primers for PCR (#O-32201, #0-32211 and #0-32212 respectively) were from TriLink Biotechnologies. Phosphate Buffered Saline (PBS, #10010-031), Dulbecco's Phosphate Buffered Saline (DPBS, #1404-133), HisPur Ni-NTA Magnetic Beads (#88831), Nunc MaxiSorp Flat-Bottom 96-well Plates (#44-2404-21), Pierce High Sensitivity Streptavidin-HRP (#21130), 1-Step Ultra TMB-ELISA (#34028), Pierce™ Nickel Coated Plates, Clear, 8-Well Strip (#15142) were purchased from ThermoFisher Scientific; High Capacity Streptavidin Magnetic Beads (#1497) were from Click Chemistry Tools; DNA Clean& Concentrator-5 (#D4013) was from Zymo Research; HS Next Generation Sequencing (NGS) Fragment kit (1-6000 bp), 500 (#5191-6578) was from Agilent Technologies; TruSeq ChiP Library Preparation Kit (#IP-202-1012) was from Illumina; Horseradish Peroxidase-conjugated Goat Anti-Mouse IgG (#115-035-062) was from Jackson ImmunoResearch Laboratories; SARS-CoV/SARS-CoV-2 (COVID-19) spike antibody [1A9] (#GTX632604) was from GeneTex; plasticware for cell culture were from CellTreat Scientific Products; Pulmonary surfactant (#THP-0147) was from Creative BioMart; ssDNA and biotinylated DNA HPLC purified were from Integrated DNA Technologies; Influenza A H1N1 hemagglutinin protein (#40005-V08H1) was from Sino Biological; SARS-CoV-2 Nucleocapsid protein (#C5227 and #230-01104) was from AcroBiosystems and RayBiotech respectively. Antibodies specific for anti-His Tag (clone: J095G46, #362605), isotype control mouse IgG2a (clone: MOPC-173, #400220), and FluoroFix Buffer (#422101) were purchased from BioLegend. Antibodies specific for anti-human ACE2 (#FAB933A) and isotype control Goat IgG (#IC108A) were purchased from R&D System. Fixable Viability Dye eFluor 450 (#65-0863-14) was purchased from Invitrogen. COVID-19 S Protein/(GFP)-(6His) VLP (#VLP001) were purchased from Gentarget, Inc.; SPRi-Biochip CSe (#1123299207) was from Horiba; all other reagents were from Sigma-Aldrich. Sequencing of the aptamers were performed on MiSeq (Illumina); the surface plasmon resonance experiments were performed using a OpenPlex SPRi system (HORIBA); Navios EX flow cytometer (Beckman Coulter) was used for flow cytometry experiments and flow cytometry data were analyzed with FlowJo v10 (FlowJo LLC).

African Green Monkey Kidney Cell (Vero E6 cells, #ATCC CRL-1586) and Eagle's Minimum Essential Medium (EMEM) (#30-2003) were obtained from ATCC; Geneticin (Antibiotic G418, #G271) was from ABM, Opti-MEM™ I Reduced Serum Medium was purchased from ThermoFisher Scientific, HEK293T cell line human (#12022001) was from Sigma-Aldrich, Dulbecco's Modified Eagle's Medium (DMEM, #10-013-CV) was purchased from Corning, plasmid ACE2 (NM 021804) Human Tagged ORF Clone (Angiotensin Converting Enzyme 2) (#RC208442) was purchased from Origene, Lipofectamine 2000 Reagent (#11668-030), StemPro Accutase (#A11105-01) and Penicillin Streptomycin (#15140-122) were purchased from ThermoFisher Scientific; Fetal Bovine serum (#SH30088.03) was from Hy-Clone.

### (2) Selection of the Aptamers for SARS-CoV-2 Trimer S Protein

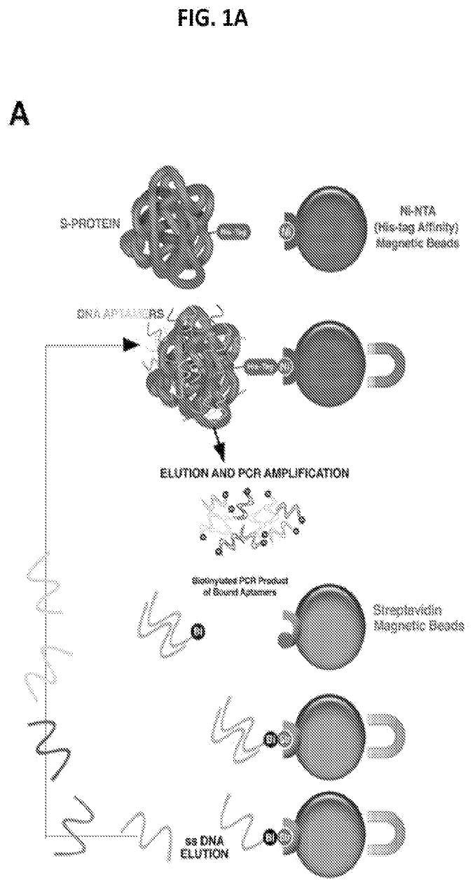

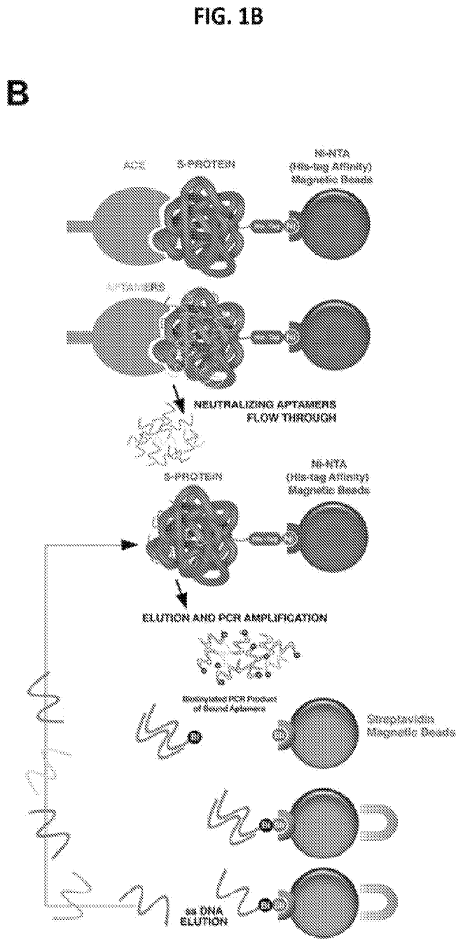

Systematic evolution of ligands by exponential enrichment (SELEX) procedure was performed. A library of single stranded DNA oligonucleotides (10¹⁵ random unique sequences) that are flanked by two 23-based primer sequences (5′ TAGGGAAGAGAAGGACATATGAT (SEQ ID NO: 161)) and (TTGACTAGTACATGACCACTTGA 3′ (SEQ ID NO: 160)) forming the formula TAGGGAAGAGAAGGACATATGAT (SEQ ID NO: 161) N40 TTGACTAGTACATGACCACTTGA (SEQ ID NO: 160); primer sequences which are also used for amplification during SELEX and thus follow the formula TAGGGAAGAGAAGGACAATGAT (SEQ ID NO: 159) N40 TTGACTAGTACATGACCACTTGA (SEQ ID NO: 160) ), where N40 is the aptamer sequence sandwiched between the 5′ and 3′ end primers were used. For the first round of SELEX, 40 μg of His-tagged SARS-CoV-2 trimer S protein was conjugated with nickel-nitrilotriacetic acid (Ni-NTA) modified magnetic beads (6 mg) by 1 hr incubation with rotation in 1 ml of washing/binding buffer A: 20 mM Na-phosphate, 0.2 M NaCl, 0.01% Tween 20 and 0.5 mM MgCl2 pH 7.4. To get the ssDNA in their unique conformation, 10 nmol of ssDNA library was heated at 95° C. for 10 min followed by 20 min incubation on ice. Activated ssDNA was pre-cleared by incubation with 6 mg of washed Ni-NTA beads to remove any ssDNA that binds non-specifically to the matrix. Two milliliters of flowthrough containing cleared ss-DNA were added to SARS-CoV-2 trimer S protein bound to Ni-NTA beads. After 15 min incubation with rotation at room temperature, beads were collected, washed three times with buffer A and captured ssDNA was eluted by 10 min incubation with 100 μl of 20 mM NaOH and neutralized with 12 μl of 0.2 M NaH2PO4 to a final of pH ˜7.2. The eluted ssDNA was amplified by PCR using biotinylated reverse primers. The following PCR program was used: polymerase activation 95° C. for 3 min, amplification at 95° C. for 20 sec, 60° C. for 20 sec and 72° C. for 20 sec repeated 17 cycles, final extension at 72° C. for 1 min. PCR product was cleaned up and concentrated using Zymo Research kit and collected dsDNA was bound to streptavidin beads in the presence of 1 M NaCl. Specific ssDNA was eluted from complementary strand with 100 μl 20 mM NaOH and neutralized with NaH2PO4. Quality of ssDNA was analyzed using Fragment Analyzer with Agilent reagents. The eluted ssDNA formed a new enriched ssDNA library pool that was used for subsequent (rolling) round of SELEX. For all following rounds of selection, the amount of immobilized S protein was decreased to 10 μg on 3 mg of Ni-NTA beads. Stringency of washing buffer A was increased to 0.5 M NaCl with a simultaneous increase in washing time up to 10 min for each wash. To remove ssDNA that nonspecifically binds to Ni-NTA matrix, negative depletion for ssDNA pool was performed after every third round of selection. To enhance the specificity of the aptamers, counter SELEX was performed using pulmonary surfactant, BSA, and human plasma. For counter selection with pulmonary surfactant, aptamers incubation with trimer S protein was performed in the presence of 0.1 mg/ml pulmonary surfactant at the 5th and 9th rounds of selection. Also, BSA and human plasma proteins were used for counter selections at 6th and 10th rounds respectively by incubation the ssDNA aptamers with immobilized S protein in the presence of 1% BSA or human plasma diluted in three times with buffer A. After six rounds of selection, the eluted ssDNA pool was used to specifically select neutralizing ssDNA aptamers (FIG. 1B). For this purpose, Human ACE2 receptors were added to trimer S protein immobilized on the beads in 6:1 molar ratio to block receptor binding site on the S protein. The eluted ssDNA from round six of selection was incubated with the proteins immobilized on the beads. The flowthrough, representing aptamers that specifically bind to S protein site concealed by ACE2 receptors were collected and proceeded for five more rounds of selection or counter selection.

### (3) Next-Generation Sequencing of Aptamers

The ssDNA pool eluted from 4th, 8th, 9th and 12th rounds of selection were subjected to library preparation for Illumina sequencing using TruSeq ChiP Library Preparation Kit from Illumina. Four pooled paired-end indexed DNA libraries were prepared, using the reagents provided in the Illumina TruSeq ChiP Sample preparation Kit, for subsequent cluster generation and DNA sequencing. Input DNA (50 ml of 200 pg/ml) was blunt-ended and phosphorylated. A single “A” nucleotide was added to the 3′ ends of the fragments in preparation for ligation to an adapter that has a single-base “T” overhang. Adapter sequences were added onto the ends of DNA to generate indexed single read or paired-end sequencing libraries. The ligation products were purified and accurately size-selected by agarose gel electrophoresis. Size-selected DNA was purified and PCR-amplified to enrich for fragments that have adapters on both ends. The final product was then quantitated before cluster generation. Sequencing was performed on Illumina MiSeq instrument. Sequence analysis was done according FASTAptamer software to identify sequences that showed enrichment across selection pools.

### (4) ELISA Based Binding Assay

To test the binding of 5′-biotinylated aptamers to trimer S protein, 100 μl of trimer S protein per well at concentration 2 μg/ml in 50 mM Na carbonate-bicarbonate buffer were adsorbed overnight in MaxiSorp plate. The plate was blocked with 1% BSA in PBS buffer for 1 hr at room temperature. 10 nM of biotinylated aptamers were added to each well and incubated with the immobilized trimer S protein in PBS buffer supplemented with 0.05% tween and 1 mM MgCl2. The same buffer was used to wash out unbound aptamers. Bound biotinylated aptamers were detected with Streptavidin-HRP and 3,3′,5,5′-tetramethylbenzidine (TMB) as a substrate. After the reaction was stopped with 2 M sulfuric acid, absorbance was measured at 450 nm. To show specific binding of 5′-biotinylated aptamers to trimer S protein, competition assays were performed using one-hundred-fold excess of the respective non-biotinylated aptamer.

### (5) Cell Culture, Transfection, and Stable Cell Line

HEK-293T cells were set up for experiments on day 0 (2×10⁵/60-mm dish) and cultured in 5% CO2 at 37° C. in Medium A (Dulbecco's modified Eagle's medium containing 100 units/ml penicillin and 100 μg/ml streptomycin sulfate) supplemented with 10% (v/v) fetal bovine serum. On day 1, the cells were washed and refed with Opti-MEM reduced serum medium and transfected with 2 μg of plasmid ACE2 (Myc-DDK-tagged)-Human angiotensin I converting enzyme (peptidyl-dipeptidase A) 2 using existing protocol from Invitrogen (Protocol Pub. No. MAN0007824 Rev1). Briefly, DNA was incubated with 6 μl of Lipofectamine 2000 in Opti-MEM reduced serum medium for 5 min at room temperature and DNA-Lipofectamine 2000 complex were added to the cells. Eight hours after transfection, cells were refed with medium A. On day 3, cells were fed with medium A containing 200 μg/ml of G418 antibiotic. Cells were maintained in this media for one week before decreasing the G418 concentration to 100 μg/ml. African Green Monkey Kidney Cell (Vero E6 cells) were set up for experiments and cultured in 5% CO2 at 37° C. in Medium B (Eagle's Minimum Essential Medium (EMEM) containing 100 units/ml penicillin and 100 μg/ml streptomycin sulfate) supplemented with 10% (v/v) fetal bovine serum.

### (6) Cell Based Inhibition Assay of SARS-CoV-2 Trimer S Protein Binding to ACE2 Receptors on the Surface of Vero E6 Cells

SARS-CoV-2 trimer S protein at 10 nM was pre-incubated with aptamers at different concentrations in 200 μl of PBS buffer containing 1 mM MgCl2 and 0.5% fish gelatin for 1 hr at room temperature. Vero E6 cells were grown in 10 cm plates to nearly 100% of confluency, washed with PBS and detached from the plate with 3 ml of StemPro Accutase by incubation at 37° C. for 20 min. Cells were collected in 15 ml centrifuge tubes and pelleted at 1,000×g for 5 min. The pellet of the cells was washed once with PBS containing 1 mM MgCl2 and 0.1% fish gelatin and resuspended in the same buffer. Suspension of cells were divided for aliquots (5×10⁶ cells per condition) in microcentrifuge tubes and pelleted again at 3,000×g for 4 min. Supernatant was carefully and completely removed and 200 μl of each condition of trimer S protein pre-incubated with aptamers was added to each pellet. The protein without aptamers in the same buffer was added as positive control. Samples were resuspended and S protein was allowed to bind to cells for 1.5 hr with occasional gentle vortexing every 15 min at room temperature. Cells with bound trimer S protein were washed out twice with PBS by centrifugation at 3,000×g for 4 min. One hundred microliters of 1% Triton X-100 in PBS were added to each pellet to solubilize cell membranes and proteins bound to them. After 1 hr incubation at room temperature with rotation insoluble cell debris were removed by centrifugation at 23,000×g for 1 hr and 100 μl of supernatant from each sample was added to each well of Nickel Coated Plate and incubated overnight to allow His-tagged SARS-CoV-2 trimer S protein to bind to the surface of the wells. Bound trimer S protein was detected with mouse monoclonal antibody against SARS-CoV-2 spike protein and Horseradish Peroxidase-conjugated Goat Anti-Mouse IgG. Each incubation with antibodies was performed in PBS buffer containing 0.05% tween, 1 mM MgCl2 and 0.5% fish gelatin for 1 hr at room temperature. After each incubation the wells were washed with the same buffer without fish gelatin three times. Bound Horseradish Peroxidase-conjugated antibodies were detected with TMB substrate. Reaction was stopped with 2 M sulfuric acid and absorbance was measured at 450 nm.

### (7) Secondary Structure Prediction

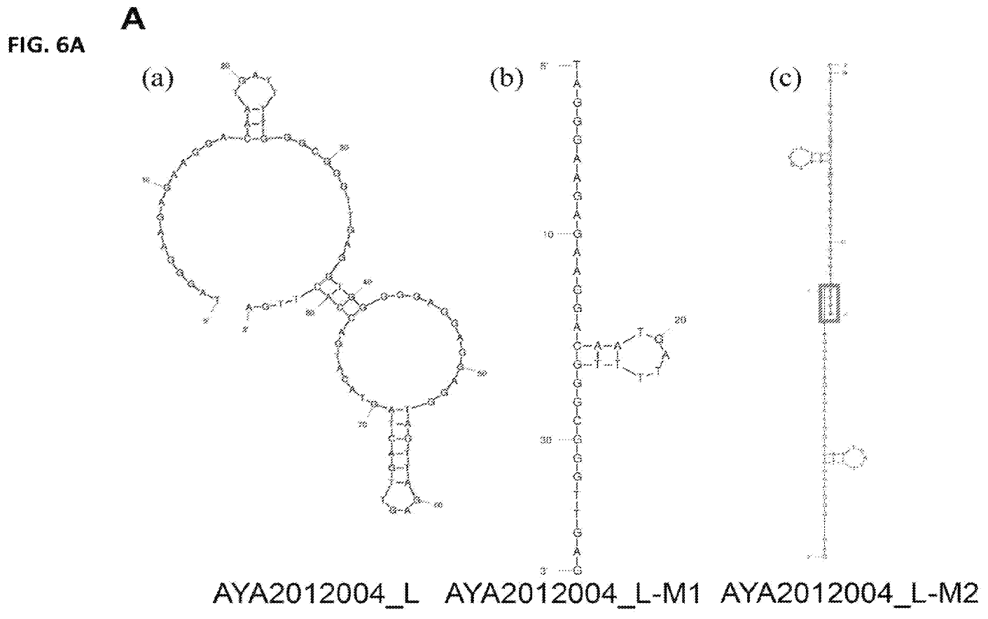

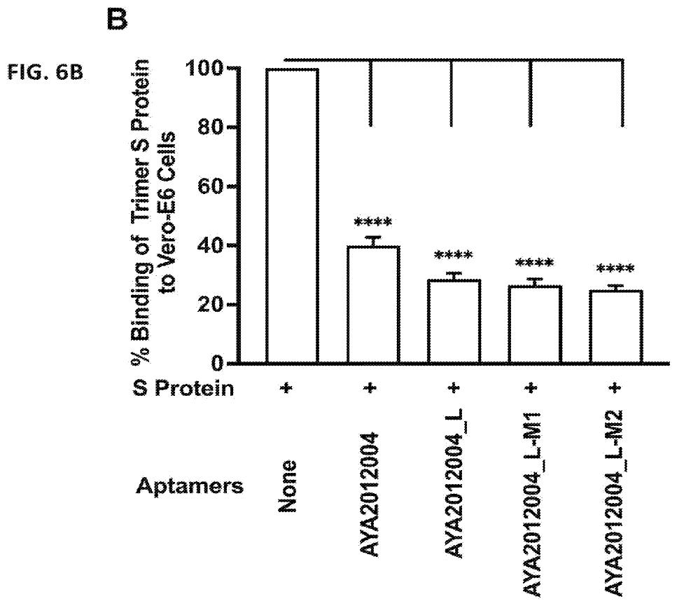

The secondary structure analysis of our aptamers was performed using the mFold webserver. A temperature of 37° C. and 0.2 M of NaCl were used for the secondary structure simulation. The secondary structure of AYA2012004 L as well as its truncated modification AYA2012004 L-M1 are shown in (FIG. 6). The truncation was performed by removing the big loop (nucleotides from position 38 to 85) from the AYA2012004 L. The AYA2012004 L-M2 was made by adding a TTTTT or AAAAA nucleotide sequences to the 5′-end of AYA2012004 L-M1 and subsequently annealing these oligos in 1:1 molar ratio, such that two copies of AYA2012004 L-M1 will have a tendency to conjugate and form a structure with two binding motifs, such a conformational change may provide enhanced inhibition.

### (8) Flow Cytometry Study of SARS-CoV-2 Trimer S Protein Binding to Vero E6 Cells and its Inhibition by Aptamers

SARS-CoV-2 trimer S protein was pre-incubated with aptamers, control aptamers with random sequences, or neutralizing antibody in PBS buffer containing 1 mM MgCl2 prior to the addition to Vero E6 cells. Trimer S protein in the same buffer alone was used as a positive control. Vero E6 cells (10⁵ cells per well) were diluted in 200 l PBS buffer and then centrifuged at 250×g for 3 min. The pre-incubated S protein at different conditions listed above was added to Vero E6 cells for 30 min at room temperature followed by two washes with PBS buffer. Anti-his-tag-APC antibodies (1 μl per 100,000 cells in 50 μl of PBS), control antibody, and fixable viability dye eFluor 450 (dilution 1:1000) were added to the corresponding wells and incubated for an additional 30 min at 4° C. followed by washing with PBS buffer. Cells were fixed with 200 μl FluoroFix buffer per well. Cells were acquired on Navios-Ex flow cytometry and data was analyzed by FlowJo v10 software.

### (9) Detection of SARS-CoV-2 Trimer S Protein Binding to Transfected 293T Cell Line Using Flow Cytometry

To confirm the expression of ACE2 receptor on the surface of stably transfected HEK293T cells, ACE2 transfected cells and mock transfected HEK293T cells were stained with anti-hACE2-APC, Goat IgG-APC, and fixable viability dye eFluor 450 at 4° C. for 30 min, followed by washing with PBS buffer. Cells were fixed with 200 μl per well FluoroFix. Fluoro fixed cells were acquired on Navios-Ex flow cytometry and data was analyzed by FlowJo v10. To demonstrate that S protein binds to ACE2 receptors on the surface of the cells, His-tagged SARS-CoV-2 trimer S protein was incubated with HEK 293T expressing ACE2 receptors and mock transfected cells at 4° C. for 30 min. After washing with PBS, cells were incubated with anti-his-tag-APC (1 μl per 10⁵ cells) or isotype control antibodies and fixable viability dye eFluor 450 (dilution 1:1000) in 50 μl of PBS at 4° C. for 30 min, cells were washed and Fluoro fixed. Cells were acquired on Navios-Ex flow cytometry and data were analyzed by FlowJo v10.

(10) Uptake of SARS-CoV-2 S Protein Expressing Virus-Like Particles (VLPs) by HEK293T Cells that Overexpress ACE2 Receptors

Mock and ACE2 receptor overexpressing HEK293T cells were cultured in 48 well plates at 60% confluency (about 10⁵ per well) in medium A supplemented with 10% FBS. VLPs that have the full-length SARS-CoV-2 S protein expressed/presented at the surface of lentiviral particle to mimic the coronavirus were used. The VLPs are packaged with Green Fluorescent Protein plasmid (GFP) as a reporter signal. VLPs (200,000 particles) were diluted in 50 μl PBS buffer in the absence or presence of aptamers and incubated at room temperature for 30 min. Preincubated VLPs of each condition were added to cultured HEK293T cells that are mock transfected or ACE2 receptor stably transfected and incubated for 72 hr. Cells were washed once with PBS and stained with viability dye eFluor 450 (dilution 1:1000) in 50 μl of PBS at 4° C. for 30 min. Uptaking of VLPs was assessed by measuring GFP signal in the cells by flow cytometry on the Navios-EX system and data were analyzed by FlowJo v10.

### (11) Binding Affinity Measurement Using Surface Plasmon Resonance (SPR)

To determine the binding affinity of aptamers to SARS-CoV-2 trimer S protein, biotinylated aptamers were immobilized as a dot in volume ˜1 μl on a CSe surface coated chip with an extravidin layer (HORIBA France, France) at a concentration of 20 μM overnight. Random biotinylated ssDNA sequences were used as a negative control. The biochip was blocked with 10 ug/ml of biotin in PBS and saturated with 1% BSA in PBS. The analytes, original strain SARS-CoV-2 trimer S protein, Delta variant of SARS-CoV-2 trimer S protein, and Alpha variant of SARS-CoV-2 trimer S protein were diluted with PBS and injected over the flow cell at concentrations of 20 nM, 5 nM, 1 nM and 0.2 nM at a flow rate of 50 μl/min with PBS as a running buffer at a temperature of 25° C. The complex was allowed to associate and dissociate for 200 sec and 360 sec, respectively. Influenza A H1N1 hemagglutinin protein, SARS-CoV-2 Nucleocapsid protein, human ACE2/ACEH protein were injected at 10 nM and serum from the healthy human donor was injected at 1:100 dilution, as negative controls. The surface was regenerated with a 200 sec injection of 1 M NaCl. Triplicate ligand spots and a buffer blank were flowed over the surface. The entire experiment was repeated on two different biochips in duplicates. The data were fit to a simple 1:1 interaction model using the global data analysis with ScrubberGen software.

(12) Docking Simulation of Interaction of Selected Aptamers with S Protein

The tertiary structures of the ssDNA aptamers were generated using the RNA composer webserver as RNA, then the RNA molecules were converted to ssDNA molecules by replacing the uracil (U) to thymine (T) and the sugar ribose in RNA to sugar deoxyribose in ssDNA using our in-house code. The converted molecules were further optimized for 5 ns using molecular dynamics. The docking simulations were performed using the PyRx virtual screening tool. The docked structures were visualized using open-source PyMOL.

### (13) Statistical Analysis

Significance was determined in Prism 9.0 (GraphPad Software) using the oneway ANOVA test (Dunnett's multiple comparisons test) or two-way ANOVA test (Tukey's multiple comparisons test) for comparisons. The p values<0.05 were considered statistically significant.

### b) RESULT

### (1) Developing High-Affinity Neutralizing DNA Apatmers Against SARS-CoV-2 Trimer S Protein

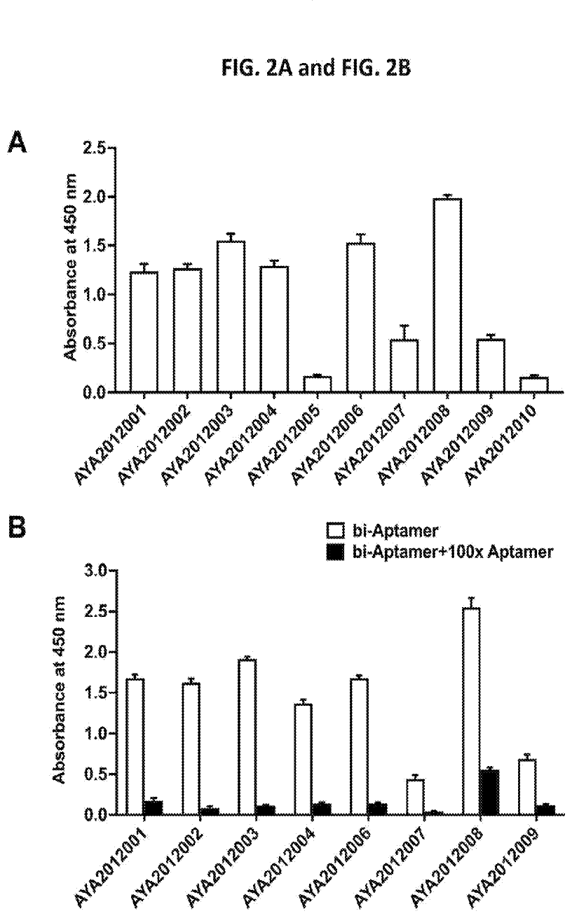

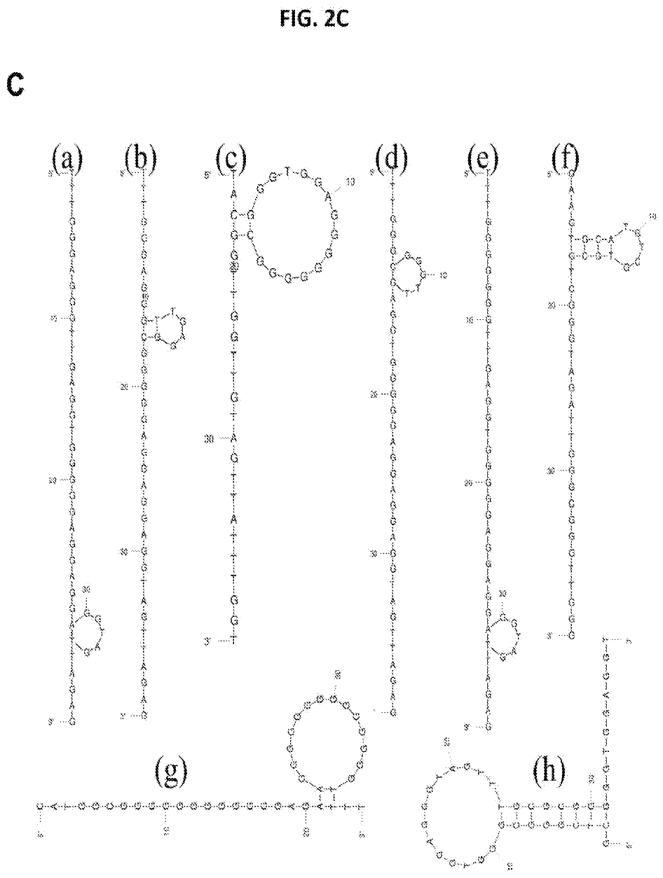

To identify single-stranded DNA aptamers that bind to SARS-CoV-2 trimer S protein and inhibit its binding to ACE2 receptors, we employed SELEX procedure as described with some modifications. SARS-CoV-2 trimer S protein that carries a polyhistidine tag at the C-terminus was immobilized on Ni-NTA magnetic beads. A library of single stranded DNA oligonucleotides (10¹⁵ random unique sequences) that are flanked by two 23-based of primer sequences was used for selection of specific aptamers. To allow ssDNA to fold into their unique conformation, 10 nmol of ssDNA library were heated at 95° C. followed by incubation on ice. Bare Ni-NTA magnetic beads were introduced to activated ssDNA library to prevent enrichment of aptamers that recognize the beads only. SELEX procedure was performed using trimer S protein coated magnetic beads as a target (FIG. 1A). The counterselection steps against Ni-NTA magnetic beads were performed after every third round of selection. The selection stringency was gradually increased by decreasing the concentration of trimer S protein, increasing the stringency of wash buffer with a simultaneous increase in washing times. After six rounds of conventional selection, human ACE2 receptor was added to trimer S protein immobilized on the beads to block the receptor binding site on trimer S protein. This complex was used to select aptamers that specifically bind to receptor binding site on trimer S protein (FIG. 2B). The eluted ssDNA from round six of selection was incubated with the proteins complex (S and ACE2 proteins) immobilized on the beads. The flowthough, representing aptamers that specifically bind to trimer S protein sites concealed by ACE2 receptors were collected and proceeded for five more rounds of selection. To enhance the specificity of the aptamers, counter selection was performed using pulmonary surfactant, human plasma, and BSA. The ssDNA pool eluted from 4th, 8th, 9th, and 12th rounds of selection were chosen for high-throughput sequencing using the MiSeq (Illumina) platform. Sequence analysis was done according FASTAptamer to identify sequences that showed enrichment across selection pools. The ten most enriched sequences after twelve rounds of selection are presented in Table 3.

TABLE 3
Most abundant aptamer sequences after twelve rounds of selection

| | | | | Reads |
|---|---|---|---|---|
| Name | Sequence | Length | Rank | (round 12) |
| AYA2012001 | TTTGGGAGGGTTGAGGTGGGGGAGGAGGAGG | 40 | 1 | 24498 |
| | TAGTTAGAG (SEQ ID NO: 35) | | | |
| AYA2012002 | TTTGGGAGGGTTGAGGCGGGGGAGGAGGAGG | 40 | 2 | 4212 |
| | TAGTTAGAG (SEQ ID NO: 37) | | | |
| AYA2012003 | TACGGGTGGAGGGGGGGGCGGTTGGTTGTAG | 40 | 3 | 1529 |
| | TTATTTGGT (SEQ ID NO: 39) | | | |
| AYA2012004 | TTTGGGCGGGTTGAGGTGGGGGAGGAGGAGG | 40 | 4 | 750 |
| | TAGTTAGAG (SEQ ID NO: 36) | | | |
| AYA2012005 | CACGTGCATGTCGTGCGTCGGGTAGATTGGGT | 40 | 5 | 400 |
| | GGGTTGGG (SEQ ID NO: 127) | | | |
| AYA2012006 | TTTGGGGGGGTTGAGGTGGGGGAGGAGGAGG | 40 | 6 | 350 |
| | TAGTTAGAG (SEQ ID NO: 40) | | | |
| AYA2012007 | CAAGTGCATGTCGTGCGTCGGGTAGATTGGGC | 40 | 7 | 254 |
| | GGGTTGGG (SEQ ID NO: 129) | | | |
| AYA2012008 | CATGGCGGGGGGGGGGGGAGAAGGGGGGGG | 40 | 8 | 200 |
| | GGGGGGTTTT (SEQ ID NO: 38) | | | |
| AYA2012009 | GCTCGGGCGGGTGGAGGGTAGTTTGCGCGGG | 40 | 9 | 197 |
| | GGTGGAGGT (SEQ ID NO: 128) | | | |
| AYA2012010 | GTGGGTGGGATATTGGTGGTGGTGCGCTAAA | 40 | 10 | 19 |
| | GTGTATTGG (SEQ ID NO: 130) | | | |

### (2) Aptamer Binding to SARS-CoV-2 Trimer S Protein.

The ten most enriched aptamer sequences were evaluated for their binding to SARSCoV-2 trimer S protein using ELISA based binding assay. Recombinant SARS-CoV-2 trimer S protein was immobilized on MaxiSorp plate and biotinylated aptamers were incubated with immobilized protein. Bound aptamers were detected using Streptavidin-HRP. All aptamers exhibited binding ability, except AYA2012005 and AYA2012010 (FIG. 2A). The specificity of the aptamers with good binding affinity were determined using competition ELISA-based binding assay (FIG. 2B). For this purpose, one-hundred-fold excess of the non-biotinylated aptamers were added to the wells with respective biotinylated aptamers. When binding is specific, nonbiotinylated aptamers compete out biotinylated aptamers and absorbance at 450 nm significantly decreases. As shown in FIG. 2B, all tested aptamers were competed out with one-hundred-fold excess of the non-biotinylated respective aptamers and demonstrated specific binding to SARS-CoV-2 trimer S protein. The predicted secondary structures of selected aptamers are shown in FIG. 2C. All selected aptamers have a hairpin structure with the unpaired loop size ranging from 4 nucleotides (AYA2012002) to 14 nucleotides (AYA2012003, AYA2012008, and AYA2012009). AYA2012001 and AYA2012006 share an almost identical predicted secondary structure, as the only difference is A/G change at site 7. The increased base pairs in AYA2012007 and AYA2012009 as compared to others can result in enhanced stability. It is worth noting that the selected aptamers are rich in G nucleotide, indicating that the formation of G quadruplex, which has been identified as a common motif in many DNA aptamers, can improve the stability of these aptamers.

### (3) Aptamers' Inhibition of the Binding of SARS-CoV-2 Trimer S Protein to ACE2 Receptors Expressed on the Surface of Vero E6 Cells

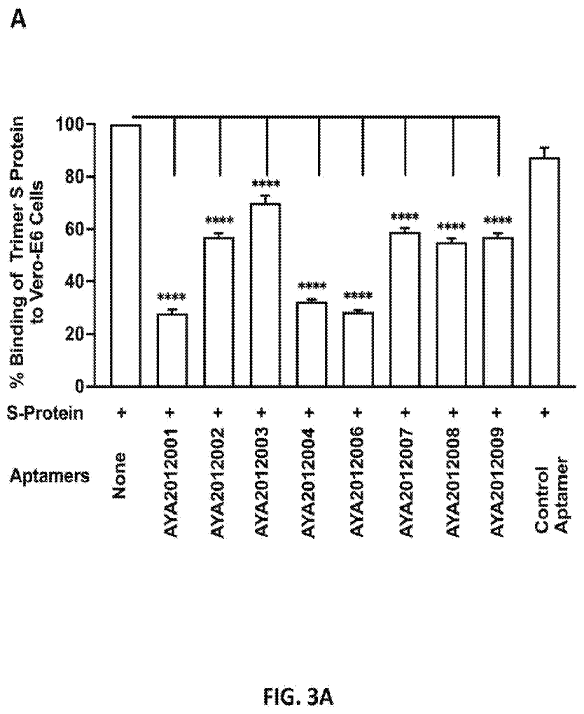

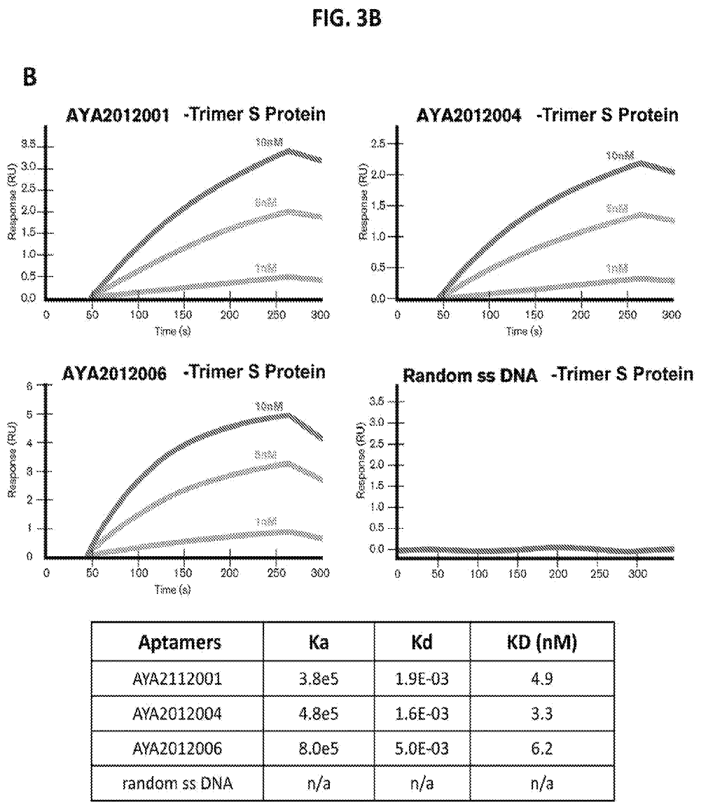

Angiotensin-converting enzyme 2 (ACE2) serves as the cell surface receptor to bind SARS-CoV-2 trimer S protein and facilitates entry of these coronaviruses into the cell. The African green monkey kidney cell line, Vero E6, are commonly used to isolate, propagate and study SARS-CoV-like viruses as they support viral entry and replication to high titers. To investigate the inhibitory effect of our aptamers on SARS-CoV-2 trimer S protein binding to ACE2 receptors, the Vero E6 cells line was used. His-tagged SARS-CoV-2 trimer S protein was incubated with Vero E6 cells in the absence or presence of the indicated aptamers. Random 40-nucleotide ssDNA was used as a control. Unbound S-protein was washed out and the cell pellet, containing bound His-tagged SARS-CoV-2 trimer S protein, was solubilized with 1% Triton X100. Insoluble cell debris was removed by centrifugation and supernatant was added to wells of Nickel Coated Plate. His-tagged S protein bound to Nickel was detected using mouse monoclonal antibodies against S protein and Horseradish Peroxidase-conjugated goat anti-mouse antibodies (FIG. 3A). Our results show that the majority of the aptamers inhibit binding of SARSCoV-2 trimer S protein to ACE2 receptors. Aptamers AYA2012001, AYA2012004 and AYA2012006 had the best inhibition efficiency up to 70%. The affinity of those aptamers was verified by surface plasmon resonance (SPR) (FIG. 3B). Aptamers AYA2012001, AYA2012004 and AYA2012006 showed a high affinity for SARS-CoV-2 trimer S-protein with Kd 4.9 nM, 3.3 nM and 6.2 nM, respectively.

### (4) Binding of Selected Aptamers to Variants of SARS-CoV-2 Trimer S Protein

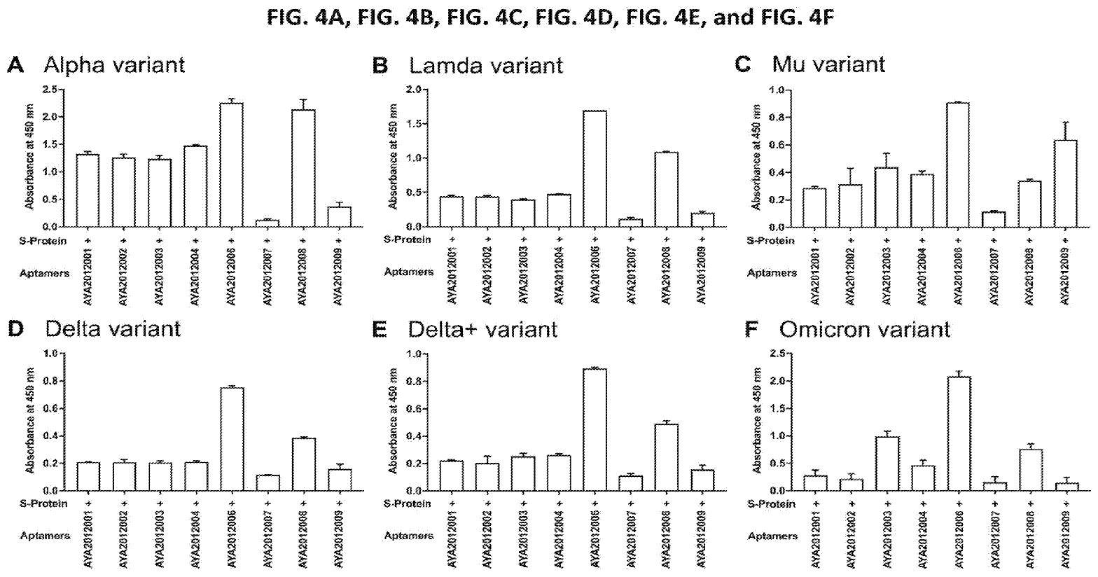

Since the pandemic started, SARS-CoV-2 virus has mutated frequently and mutations in the SARS-CoV-2 S proteins have conferred an advantage to the virus. For example, the N501Y mutation in B.1.1.7 (Alpha), T487K, P681R, and L452R mutations in B.1.617.2 (Delta), K417N mutation in AY.1/AY.2 (Delta plus), D614G, P681H, and D950N mutations in B.1.621 (Mu), G75V, T76I, A246-252, L452Q, F490S, D614G, and T859N mutations in C.37 (Lambda), T478K, Q498R, and H655Y mutations in B.1.1.529 (Omicron) have been shown to majorly contribute to the virus's ability to become more infectious, bind more tightly to human cells ACE2 receptors, and evade vaccines and some neutralizing antibodies. In this study, ELISA-based binding assay was performed to determine if selected aptamers could recognize the mutated S proteins of the COVID-19 variants (FIG. 4). Recombinant SARS-CoV-2 trimer S proteins from (A) Alpha, (B) Lambda, (C) Mu, (D) Delta, (E) Delta plus, and (F) Omicron variants were immobilized on MaxiSorp plate. Biotinylated aptamers were added and bound aptamers were detected using Streptavidin-HRP. As seen in FIG. 4, our aptamers demonstrated different binding affinities to the SARS-CoV-2 trimer S protein variants, with AYA2012006 and AYA20122008 showing the best binding to all variants and aptamer AYA2012009 showing the best binding to Mu compared to other tested variants. This presents the advantage of potentially developing combined therapy consisting of aptamers that can target different variants of the S protein.

### (5) Long Aptamers Bind to SARS-CoV-2 Trimer S Protein and Inhibit the S Protein Binding to Vero E6 Cells

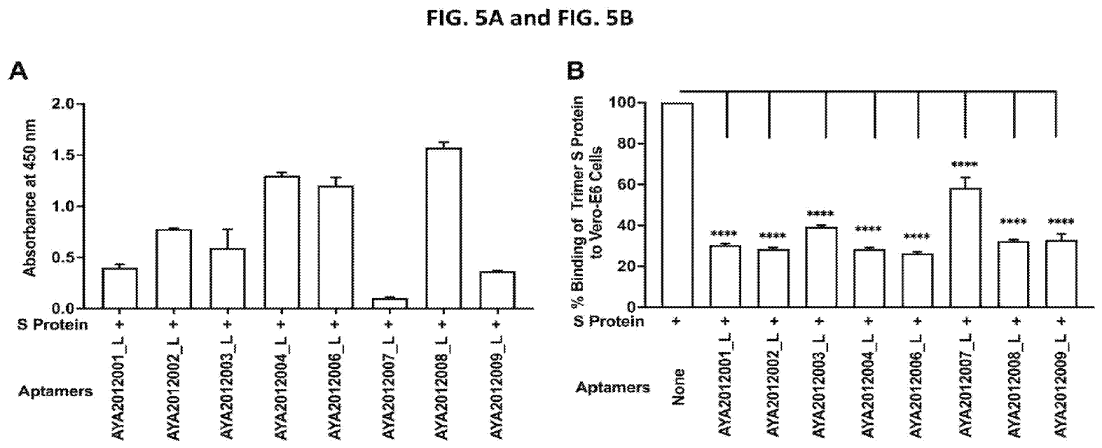

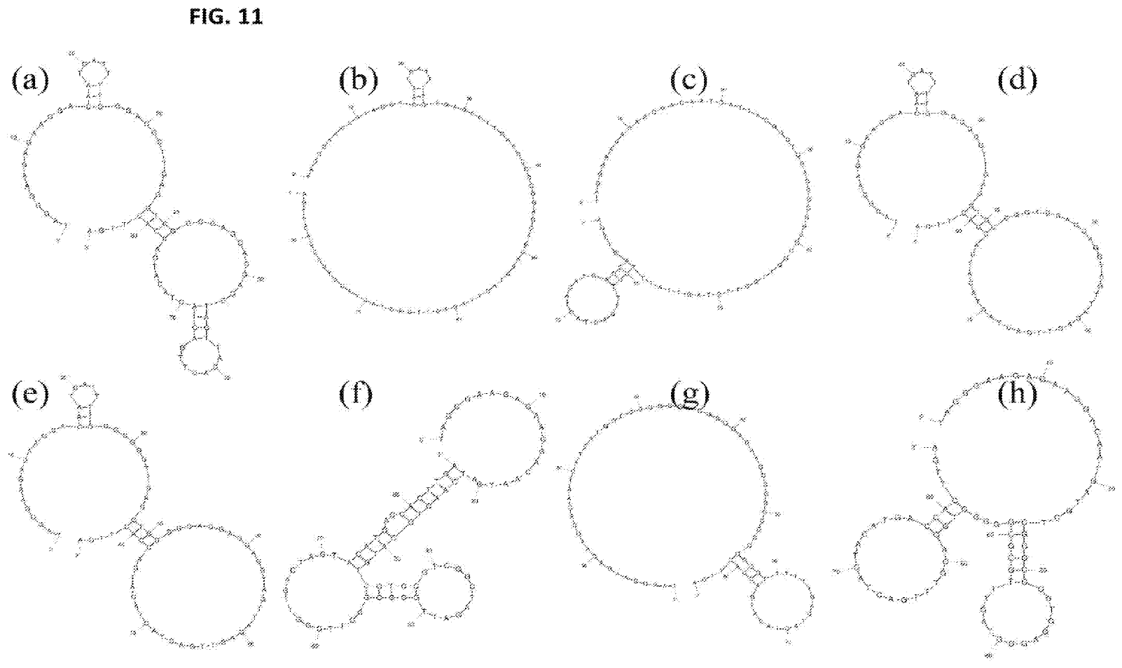

Candidate aptamers containing primer sequences (long aptamers) were tested for their binding ability to SARS-CoV-2 trimer S protein and inhibitory effect on SARSCoV-2 trimer S protein binding to ACE2 receptors on Vero E6 cells (FIG. 5). The experiments were performed. All long aptamers except AYA2012007 L demonstrated good binding ability as seen in FIG. 5A. Among the long aptamers, AYA212001 L, AYA 212002 L, AYA2012004 L, and AYA2012006 L showed better inhibitory activity (FIG. 5). It is valuable to note that aptamer AYA2012004 L provided the best balance between binding ability and inhibition efficiency. The predicted secondary structure of long aptamers are shown in FIG. 11. Based on the predicted structures, the incorporation of the two conserved primer sequences changed the secondary structures of all aptamers. For example, an additional hairpin structure with 36 unpaired nucleotides along with a 4 base pairs of stem appears in AYA2012004 L. Among the 8 selected aptamers, AYA2012001 L, AYA2012004 L, AYA2012006 L, AYA2012007 L and AYA2012009 L appeared to have a divalent structure (two hairpin motifs). The additional pairs in the stems of these long aptamers are expected to enhance their stability.

### (6) Characterization of Modified Aptamers

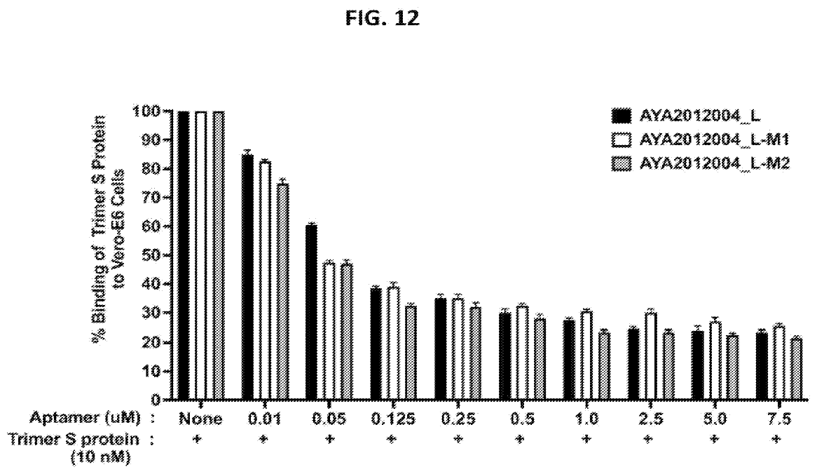

Since aptamer AYA2012004 L demonstrated the best balance between binding ability and inhibitory efficiency, we decided to further modify and enhance its efficacy. To access the importance of different motifs in AYA2012004 L, we truncated its secondary structure into different motifs. The structures of modifications are shown in FIG. 6A. Aptamer AYA2012004 L-M1 is modified by removing the big loop (nucleotides from position 38 to 85) from the AYA2012004 L. Aiming to further enhance the binding and inhibition, a bivalent structure of AYA2012004 L-M2 was further constructed by adding an additional AAAAA or TTTTT to AYA2012004 L-M1, such that the two aptamers with poly A and poly T could have a tendency to A-T paring through the AAAAA-TTTTT pairs and function as a system with two binding centers. The interaction between AAAAA-TTTTT can also provide additional inhibition because of the steric effects. The inhibitory efficiency of AYA2012004 L and its modified versions was tested (FIG. 6B). Interestingly, AYA2012004 L-M1, which is the truncated AYA2012004 L after trimming out the bigger hairpin structure (38-85), remains almost as effective as AYA2012004 L with respect to binding and inhibition. The AYA2012004 L-M2 demonstrated strong inhibitory activity as well. Concentration dependence of inhibition of trimer S protein binding to Vero E6 cells was obtained for all three modified aptamers and results are shown in FIG. 12. Modified aptamers AYA2012004 L-M1 and AYA2012004 L-M2 demonstrated up to 70% inhibition of trimer S protein binding to Vero E6 cells even at concentration as low as 0.05 M.

(7) Binding Affinity and Specificity Determination of Modified Aptamers to Different Variants of S Protein with Surface Plasmon Resonance (SPR)

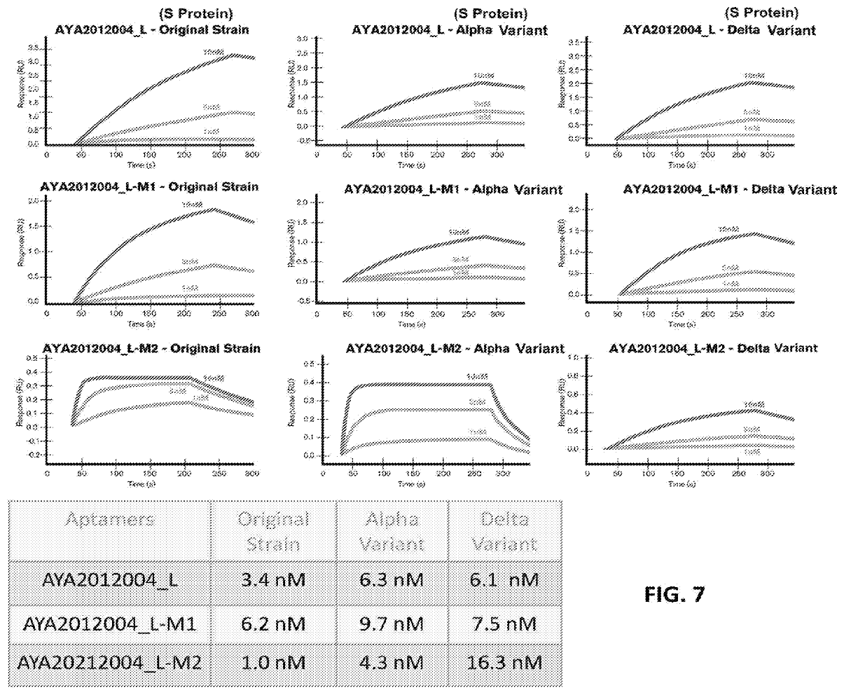

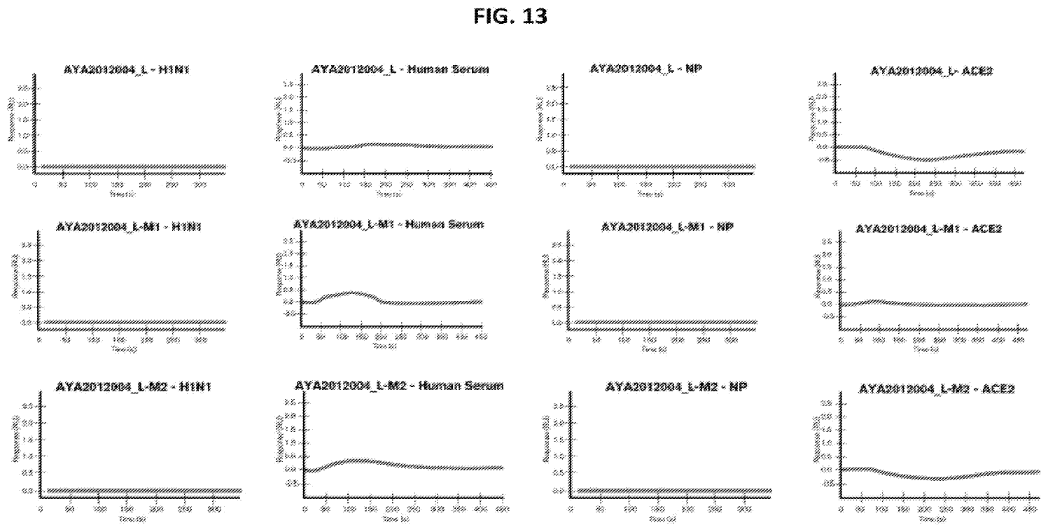

The selected aptamers AYA2012004 L, AYA2012004 L-M1 and AYA2012004 L-M2 were further characterized for their binding affinity to SARS-CoV-2 trimer S protein of original strain, Delta, and Alpha variants with surface plasmon resonance. Interestingly, all of the selected aptamers showed a high binding affinity for these variants' trimer S proteins in nM range. The Kd numbers were determined and are presented in the table (FIG. 7). The modified aptamer AYA2012004 L-M2 showed a high binding capacity to SARS-CoV-2 trimer S protein of original strain and Alpha variant with Kd 1 nM and 4.3 nM, respectively. Specificity of the selected aptamers was evaluated by measuring their binding to the following four proteins: Influenza H1N1 hemagglutinin proteins, SARS-CoV-2 nucleocapsid protein, Human ACE2/ACEH protein, and serum from a healthy human donor. FIG. 13 shows SPR data for all three aptamers. The data clearly indicated that none of the four control proteins showed detectable binding to either aptamer. The result showed that all three aptamers recognized SARS-CoV-2 trimer S protein with high affinity and specificity.

### (8) Flow Cytometry Based Assay to Assess the Inhibitory Effect of Modified Aptamers on the Binding of Variants of Trimer S Protein to ACE2 Receptors on Vero E6 Cell

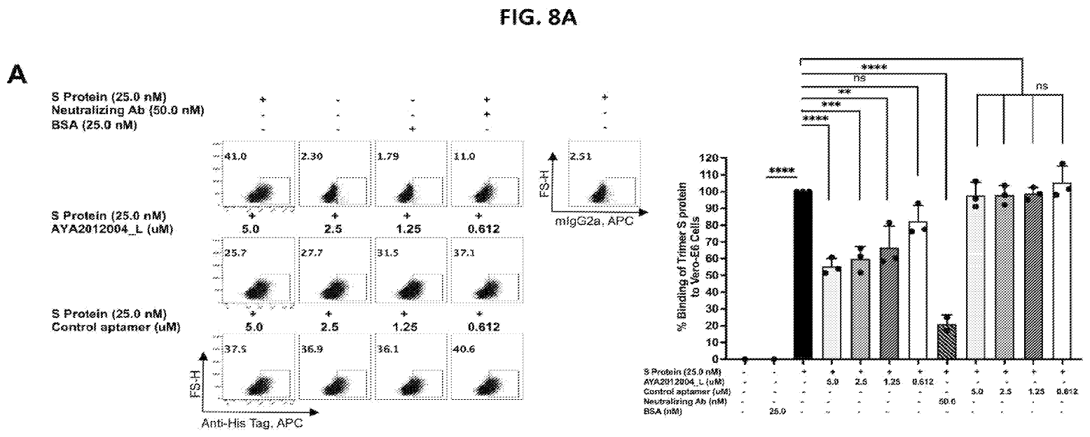

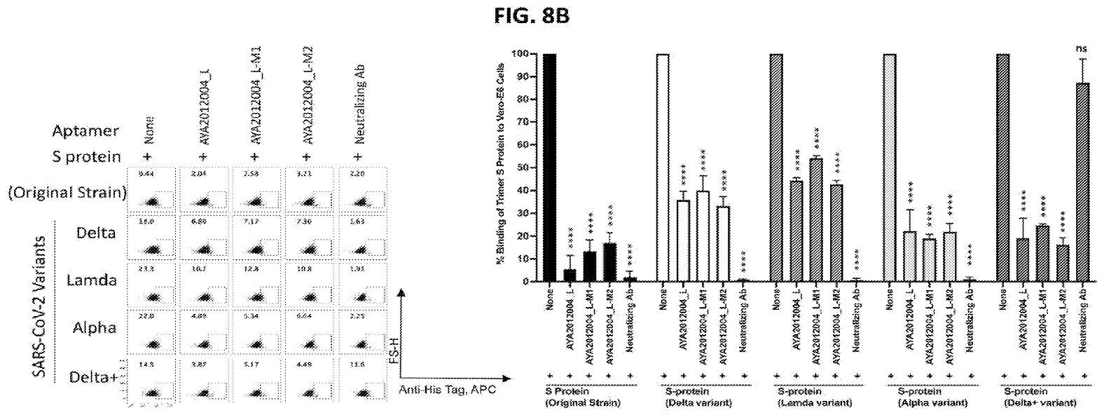

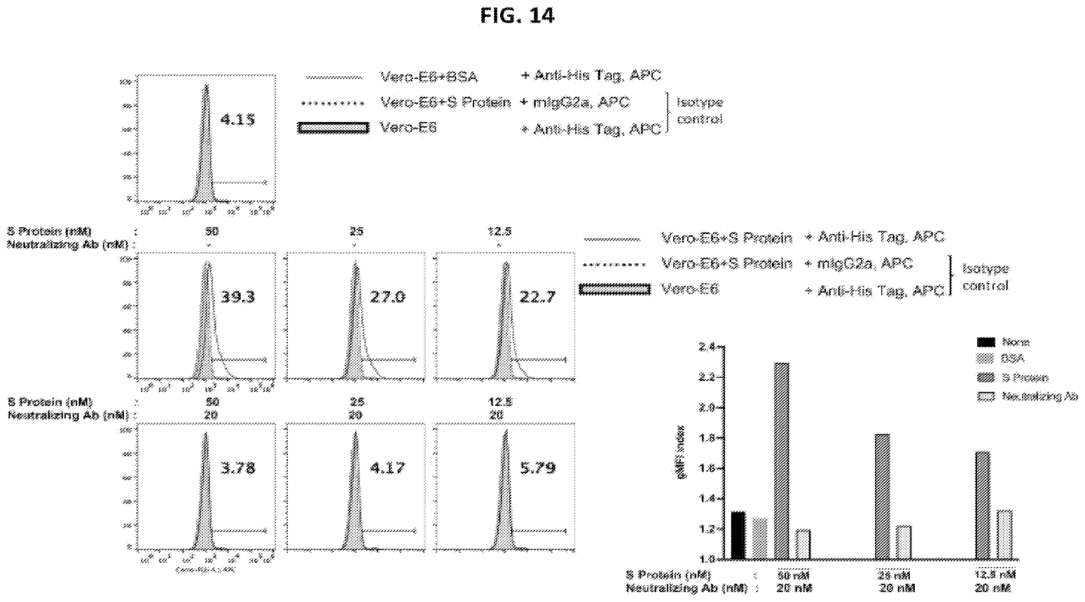

Flow cytometry approach was used to confirm SARS-CoV-2 trimer S protein binding to ACE2 receptors on the surface of Vero E6 cells. Binding of trimer S protein to Vero E6 cells was determined at various concentrations of S protein as indicated in FIG. 14. Specificity of trimer S protein binding was confirmed by the neutralizing antibodies that inhibit trimer S protein binding to ACE2 receptors at two fold excess. Trimer S protein at 50 nM, 25 nM and 12.5 nM concentrations binds by approximately 39%, 27% and 22%, respectively. Binding percentage was also confirmed on gMFI index that shows concentration dependent binding. To test the inhibitory efficiency of AYA2012004 L aptamer, SARS-CoV-2 trimer S protein was pre-incubated with different concentrations of the aptamer before addition to Vero E6 cells. Concentration dependent inhibition of the trimer S protein binding to Vero E6 cells by aptamer AYA2012004 L is demonstrated in FIG. 8A. Aptamer concentration 5 μM and trimer S protein concentration 12.5 nM were thus chosen for further inhibition experiment for trimer S protein from different strains binding to Vero E6 cells. Aptamers AYA2012004 L, AYA2012004 L-M1, and AYA2012004 LM2 were selected to determine their neutralizing effect on various SARS-CoV-2 trimer S proteins binding to ACE2 expressed Vero E6 cells. Trimer S proteins binding to ACE2 receptors on the surface of Vero E6 cells was significantly neutralized by aptamers AYA2012004 L, AYA2012004 L-M1, and AYA2012004 L-M2 at concentration 5 uM as indicated (FIG. 8B). Notably, anti-SARS-CoV-2 RBD neutralizing Ab inhibits interaction between S protein of Wuhan origin strain, Delta, Lambda, and Alpha and ACE2 receptors, however, anti-SARS-CoV-2 RBD neutralizing Ab was not able to inhibit interaction between S protein of Delta plus variant and ACE2 receptors. Taken together, these results show that aptamers, AYA2012004 L, AYA2012004 L-M1, and AYA2012004 L-M2 bind to trimer S protein of various strains of SARS-CoV-2 and inhibit the binding of these trimer S proteins to ACE2 receptor expressed Vero E6 cells.

### (9) Selected Aptamers and Omicron

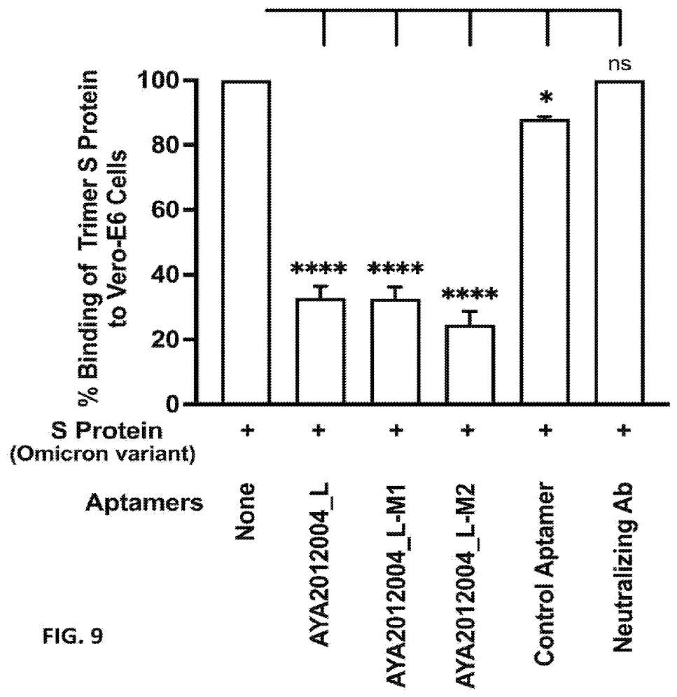

The Omicron variant of SARS-CoV-2 has recently emerged and gradually become the major variant spreading through communities. The Omicron variant has a total of 60 mutations compared to the ancestral variant, 32 of which affect the S protein, and 15 of those that are located in the receptor binding domain. This resulted in several antibodies that target the S protein RBD to become less effective or ineffective, which created a new concern for the public health system and medical care. Our aptamers AYA2012004 L, AYA2012004 L-M1, and AYA2012004 LM2 were tested for their inhibitory efficiency for SARS-CoV-2 Omicron trimer S protein binding to ACE2 receptors on the surface of Vero E6 cells (FIG. 9). Our results show that our modified aptamers' inhibition of the binding of Omicron trimer S protein to ACE2 receptors is similar to other S protein variants. Notably, the anti-SARS-CoV-2 RBD neutralizing antibody was not able to inhibit the binding of SARS-CoV-2 trimer S protein to ACE2 receptors when tested at 50 nM concentration.

(10) Neutralizing Aptamers Prevent Entry of S Protein Virus-Like Particles (VLPs) into HEK293T Cells Expressing ACE2 Receptor

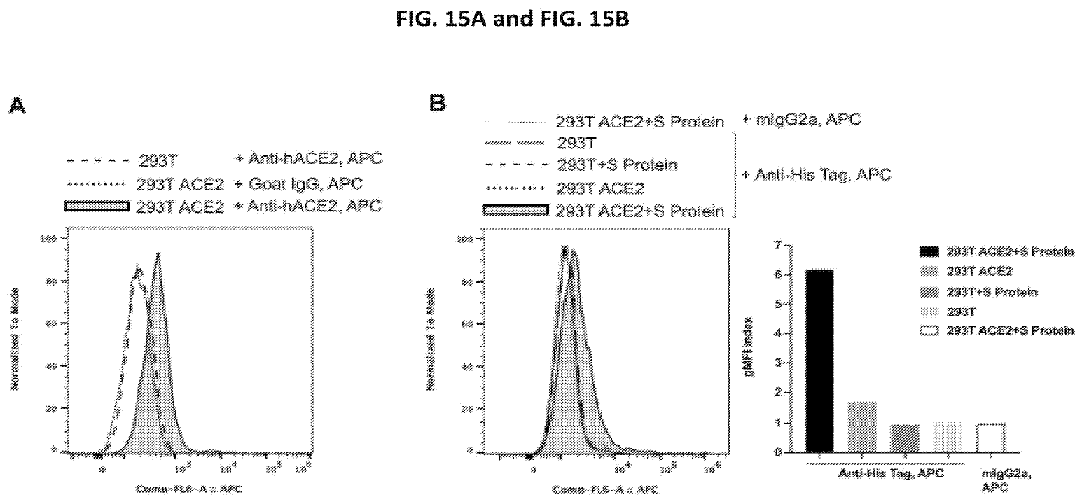

Stably transfected Human Embryonic Kidney 293 T cells (HEK-293T cells) expressing ACE2 receptor were obtained using standard protocol from Invitrogen. ACE2 expression on the surface of transfected HEK293T cells was confirmed with Flow cytometry (FIG. 15A). ACE2 receptor HEK293T cells were stained with anti-hACE2-APC. We observed a shift of fluorescent peak for transfected cells as compared to mock transfected HEK293T cells (FIG. 15A), confirming the expression of ACE2 receptor on the surface of the transfected cells. To demonstrate that SARS-CoV-2 trimer S protein binds to ACE2 receptors on the surface of the cells, His-tagged SARS-CoV-2 trimer S protein was incubated with HEK 293T cells expressing ACE2 receptor and mock transfected cells, followed by washing unbound trimer S protein. Bound trimer S protein was detected with anti-his-tag-APC antibodies (FIG. 15B). The result demonstrated that SARS-CoV-2 trimer S protein specifically binds to ACE2 receptor overexpressing cells.

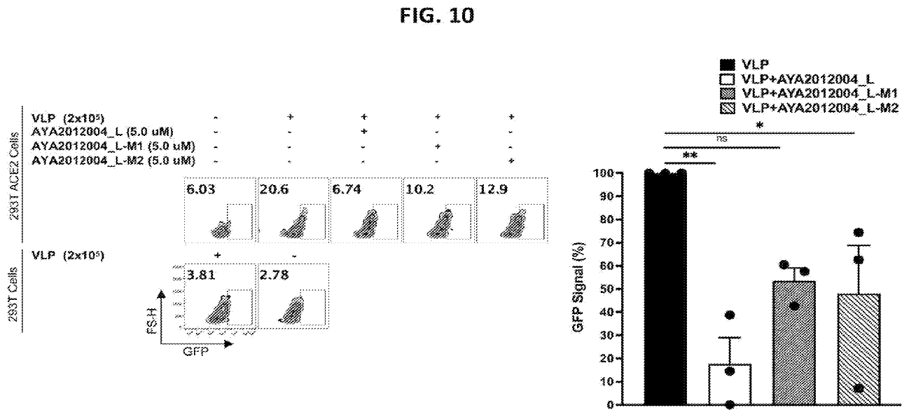

The above cell culture model was used to study the inhibitory activity of our selected aptamers on the uptake of VLPs. In this study, we used VLPs that have the full-length SARS-CoV-2 S protein expressed/presented on the surface of lentiviral particles to mimic the coronavirus. The VLPs are packaged with Green Fluorescent Protein (GFP) plasmid as a reporter signal. When VLPs bind to the cells that express ACE2 receptors, GFP plasmid can enter the cell's cytoplasm and signal can be detected after GFP protein is expressed by HEK293T cells. FIG. 10 shows that the GFP signal is detected in 20% of ACE2 receptor expressing stable cell line as compared to 3% of mock transfected cell that show background signal. In other words, our VLPs were taken up by 20% of the ACE2 receptor expressing cells though binding to S protein. Additionally, all VLPs expressing S protein significantly decreased the binding and subsequent uptake of VLP by approximate 70% in the presence of aptamer AYA2012004 L, 50% in the presence of AYA2012004 LM1, and 60% in the presence of AYA2012004 L-M2 (FIG. 10). The data presented herein indicate that the modified aptamers, through their binding to the S protein expressed on the VLP surface, inhibit its interaction with the ACE2 receptors expressed on the cell surface and prevent viral entry.

### c) Discussion

Using the SELEX process, we identified a series of neutralizing DNA aptamers that bind with high specificity and affinity to the trimer S protein, which binds to the ACE2 receptor expressed on the surface of cells. The specificity and binding affinity of aptamers are highly dependent on the three-dimensional structure of their target molecule, which is affected by the conditions implemented during the SELEX process. Unlike other studies that developed aptamers utilizing the RBD domain of the S protein as a target, making it difficult to develop neutralizing aptamers that recognize ACE2 receptors binding site, we performed the SELEX against the trimer S protein which is the closest to the native conformation of the target as it is expressed on the envelope of the SARS-CoV-2 virus. The trimer S protein of SARS-CoV-2 binds to ACE2 receptors, the host cell surface receptor, and mediates subsequent viral entry via membrane fusion. The selected aptamers were identified after 12 rounds of selection that included stringent wash conditions as well as a series of counter selections. Counter selection against blocked beads eliminated nonspecifically bound aptamers whereas counter selection against the trimer S protein-ACE2 complex enriched for aptamers that bind to the RBD domain that is concealed by ACE2 (neutralizing aptamers). The first step of viral infection is the entry of the virus to the host cell. In the case of COVID-19 disease, this is initiated by the binding of the S protein that is expressed on the envelope of the SARS-CoV-2 virus to the ACE2 receptor expressed on lung alveolar epithelial cells.

Since aptamers used during the SELEX process consist of 40 variable nucleotides that are flanked by the 23-based conserved sequences on each side, we wanted to test if the additional primer sequences could affect the secondary structure and therefore the binding and subsequent inhibitory properties of our aptamers. Interestingly, adding the primer sequences to aptamers AYA2012004 increased the binding and inhibitory activity of the aptamer.

Aptamers are more stable when they are shorter in length. The truncation of AYA2012004 L was performed using a similar method implemented by a study done by Li et al. where the hairpin structures and the unpaired nucleic acids sequences are truncated out sequentially from the entire structure. Among the truncated aptamers, AYA2012004 L-M1 appeared to have comparable affinity and inhibition to Wuhan original strain S protein as compared to AYA2012004 L, while having only 37 nucleic acids as compared to 85 of AYA2012004 L. ‘AAAAA’ and ‘TTTTT’ nucleotide sequences were further added to AYA2012004 L-M1, such that two molecule of AYA2012004 L-M1 were expected to have a tendency to form a duplex structure. We noticed very recently Zhang et al. also adopted a dimeric strategy to enhance the affinity by using multiple T sequence to connect two binding aptamers motifs.

This study led to the development of a series of neutralizing aptamers that bind to the target trimer S protein in the nM range. Our aptamers depicted different binding affinities to the variants tested. Developing a drug consisting of a combination of aptamers can confer an advantage since it can target different variants of SARS-CoV-2 S protein resulting in a universal drug for all COVID variants.

Our aptamers are specific for SARS-CoV-2 S protein since they did not display any binding to Influenza H1N1 hemagglutinin proteins, SARS-CoV-2 Nucleocapsid protein, or plasma proteins. Modified Aptamers AYA2012004 L, AYA2012004 LM1, and AYA2012004 L-M2 blocked the binding of the trimer S protein to ACE2 receptors expressed on the surface of VeroE6 cells as determined by ELISA based assay as well as flow cytometry using anti-S protein antibodies.

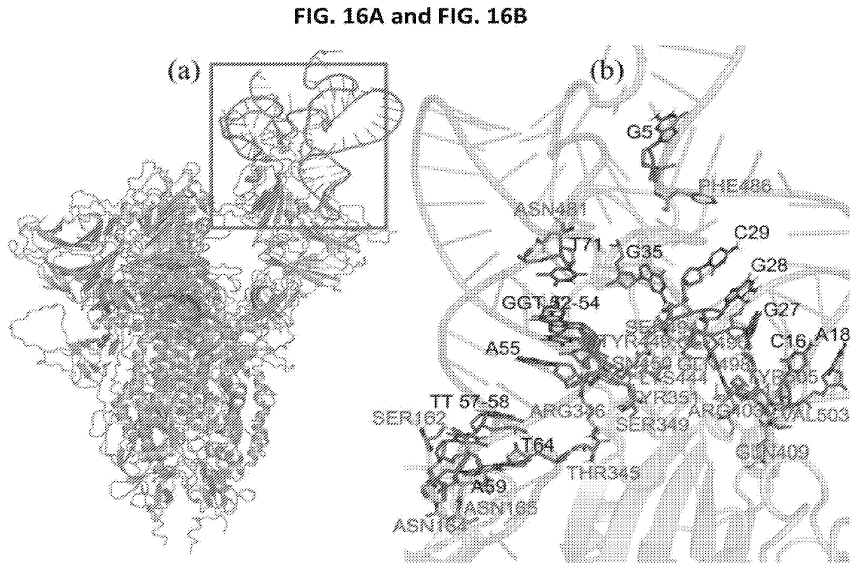

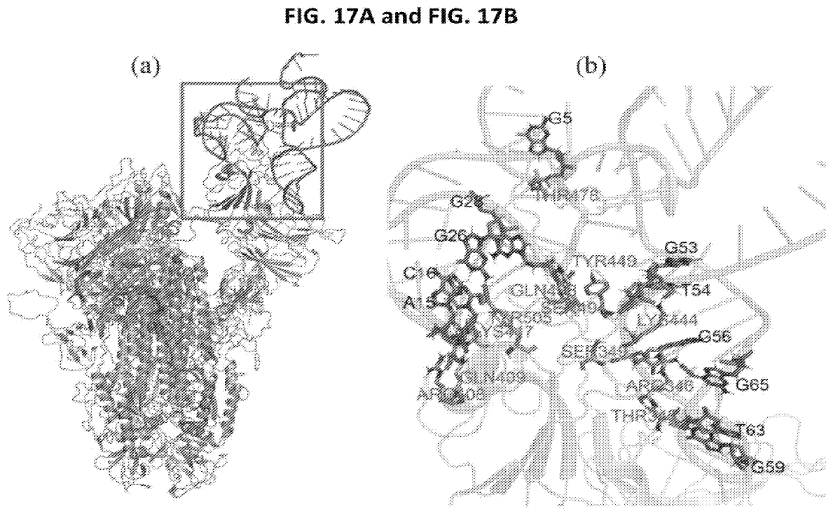

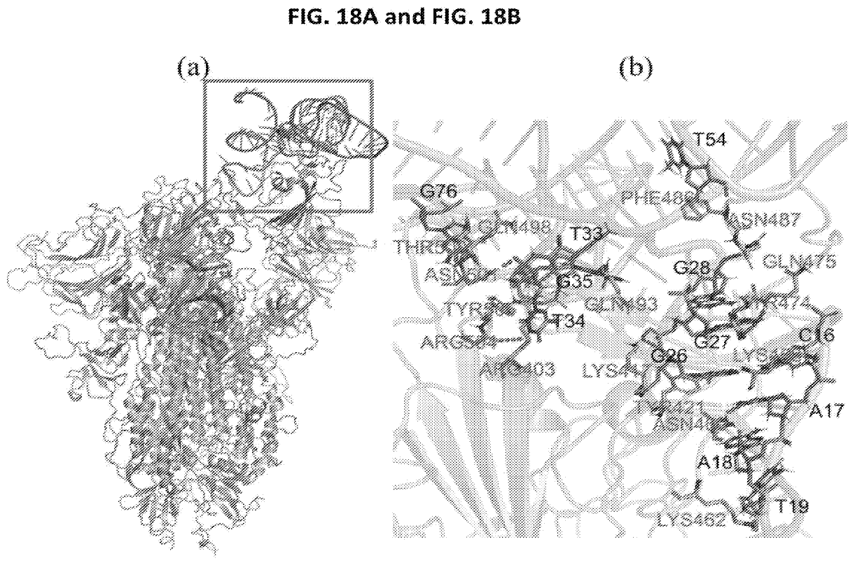

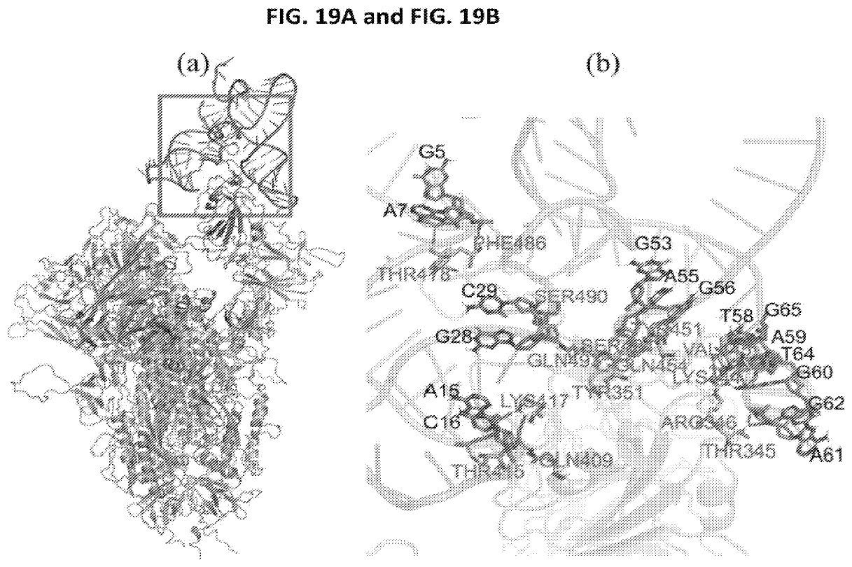

Recently, the Omicron variant was identified and included 60 mutations, many of which are new to this variant and are in the RBD of S protein. As variants of concern for SARS-CoV-2 virus continue to emerge, the concern about the efficacy of available therapies continues to rise. Our neutralizing aptamers hold the potential to substitute antibody used in COVID-19 therapy not only due to cheaper production cost, scalability, very low immunogenicity, no lot-to-lot variation, and higher shelf life stability but also because they can bind to the Delta, Mu, Alpha, Lambda, Delta plus, and Omicron variants of the trimer S protein and inhibit the binding of the different S protein variants to the ACE2 receptors. In fact, we show that the anti-SARS-CoV-2 RBD neutralizing antibody failed to block the interaction between the Delta trimer S protein (FIG. 8) and Omicrom trimer S protein (FIG. 9) and ACE2 receptor whereas aptamers AYA2012004 L, AYA2012004 LM1 and AYA2012004 L-M2 displayed more than 70% inhibition. The finding of our aptamers being able to bind to and inhibit the binding of different variants of S proteins to ACE2 receptors regardless of the mutations is also supported by our molecular docking simulations, as shown in FIGS. 16-19, where the docking complex of our champion aptamer AYA2012004 L on Wuhan original strain, Alpha, Delta, and Lambda S protein variants are displayed. It can be found that the interaction between AYA2012004 L and S proteins are very diverse, for example, more than 12 amino acid residues in the RBD domain of the S proteins are involved in forming hydrogen bonds with AYA2012004 L. We suspect the diversity of the interactions between our aptamer with the S protein make our aptamers less vulnerable to the mutations occurring in the different variants. Since the strong binding is retained in all of the S protein variants in this study, the attached aptamers prevent the interactions between ACE2 receptor and S protein, which results in the good inhibition observed in this work.

A number of studies have reported and chosen DNA aptamers to bind RBD, S1 protein, or trimerized S protein aimed at developing COVID therapies or diagnostic tools. In these studies, their DNA aptamers were found to have high affinity and inhibitory activity. To confirm the uniqueness of our aptamers, the similarity analysis was conducted comparing our selected aptamers and 77 other aptamers from 10 different publications. Heatmap plots in showed similarity scores are below 0.75, indicating our aptamers are unique as compared to the published ones. Moreover, our aptamers are rich in G, indicating the stability of these aptamers can be further enhanced with the formation of G quadruplex, which has been reported as a common motif in many DNA aptamers.

Our aptamers can be a potential therapy for COVID-19 due to their stability, ability to recognize different variants of S protein, and ability to prevent viral uptake by inhibiting the binding of S protein to ACE2 receptors.

To address the limitation of current COVID-19 treatment therapy due to the continuous emergence of new SARS-CoV-2 variants of concern, we developed a series of single-stranded DNA (ssDNA) aptamers that were not only able to bind to the trimer S protein of the Wuhan original strain, but also bind multiple variants of trimer S proteins of Delta, Delta plus, Alpha, Lambda, Mu, and Omicron. Our selected aptamers inhibited the binding of variants of trimer S protein to ACE2 receptors in Vero E6 cell line. Furthermore, our modified aptamers AYA2012004 L, AYA2012004 L-M1, and AYA2012004 L-M2 showed up to 70% inhibition of the binding and uptake of virus-like particles (VLPs) expressing S protein to ACE2 receptor expressed in Human Embryonic Kidney 293T (HEK293T) cells that overexpress ACE2 receptors. Overall, the findings indicate that our reported aptamers are an innovative therapy for the treatment of COVID-19. They hold many advantages over existing therapies due to better efficacy, the ability to identify different variants of SARS-CoV-2 and safety.

1. An isolated nucleic acid comprising sequence as set forth in any of SEQ ID NOs: 1-158 and/or 162-170, or any fragment or variant thereof comprising at least 87% sequence identity thereto.

2. The isolated nucleic acid of claim 1, wherein the sequence comprises SEQ ID NO: 35, SEQ ID NO: 36, SEQ ID NO: 37, SEQ ID NO: 39, SEQ ID NO: 127, SEQ ID NO: 40, SEQ ID NO: 129, SEQ ID NO: 38, SEQ ID NO: 128, SEQ ID NO: 130, SEQ ID NO: 66, 65, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, or SEQ ID NO: 170.

3. The RNA equivalent of any of the nuclei acids of claim 1.

4. The isolated nucleic acid of claim 1, further comprising a detectable tag.

5. The isolated nucleic acid of claim 4, wherein the detectable tag comprises a latex bead, magnetic bead, fluorescence label; fluorescent probe, chemiluminescent labels, radiolabels, and/or nanoparticle probe.

6. A composition comprising one or more of the isolated nucleic acids of claim 1.

7. The composition of claim 6, further comprising a nanoparticle or hydrogel, wherein the isolated nucleic acid is contained within the nanoparticle or hydrogel.

8. A kit comprising one or more of the nucleic acids of claim 1.

9. A method of detecting a viral infection in a subject comprising obtaining a biologic sample from the subject and measuring the concentration of a viral protein in the subject using one or more of the nucleic acids of claim 1.

10. The method of claim 9, wherein the viral infection is selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

11. The method of claim 9, wherein the viral protein is a coronavirus S protein.

12. The method of claim 11, wherein the coronaviral S protein comprises an S protein from avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2), SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, A246-252, L452Q, F490S, D614G, and T859N mutations in C.37 (Lambda), SARS-CoV-2 with T478K, Q498R, and H655Y mutations in B.1.1.529 (Omicron), and middle east respiratory syndrome (MERS) coronavirus (CoV) (MERS-CoV).

13. A method of treating, inhibiting, reducing, decreasing, ameliorating, and/or preventing a viral infection in a subject comprising administering to the subject one or more of the nucleic acids of claim 1.

14. The method of claim 13, wherein the viral infection is selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

15. The method of claim 14, wherein the coronavirus comprises an avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2), SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, A246-252, L452Q, F490S, D614G, and T859N mutations in C.37 (Lambda), SARS-CoV-2 with T478K, Q498R, and H655Y mutations in B.1.1.529 (Omicron), and middle east respiratory syndrome (MERS) coronavirus (CoV) (MERS-CoV).

16. The method of claim 15, wherein the nucleic acid binds to the S protein of the coronavirus.

17. The method of claim 16, wherein the nucleic acid binds to the S protein in such a manner as to inhibit the binding of the S protein to the ACE2 receptor.

18. The method of claim 11, wherein the one or more nucleic acids are selected from the group consisting of AYA2012001, AYA2012002, AYA2012003, AYA2012004, AYA2012005, AYA2012006, AYA2012007, AYA2012008, AYA2012009, AYA2012010, S1, S1L, S2L, S4, S5, S9, S10, S11, S12, SB1L, SB3L, SB5L, SB16, RR68, RR74, RR80, RR83, RR84, RR87, RR92, and RR93.

19. (canceled)

20. (canceled)

21. (canceled)

22. (canceled)

23. The method of claim 9, wherein the one or more nucleic acids are selected from the group consisting of AYA2012001, AYA2012002, AYA2012003, AYA2012004, AYA2012005, AYA2012006, AYA2012007, AYA2012008, AYA2012009, AYA2012010, S1, SlL, S2L, S4, S5, S9, S10, S11, S12, SB1L, SB3L, SB5L, SB16, RR68, RR74, RR80, RR83, RR84, RR87, RR92, and RR9.
